SEQUENCE LISTING (9 sequences)

SEQ ID NO: 1 - DNA, 720 bp, synthetic construct
  misc_feature 1..720: note = Codon-optimised CLE1 gene
  source 1..720: mol_type = other DNA
atgttggtttcagcattggctttagcagttttgtctgctgcatcattaggtagagctgcaccaacaccagaatctgctgaagcacatgaattggaagctagagcaacttcttcagcttgtccacaatacgttttgattaatacaagaggtactggtgaaccacaaggtcaatcagctggttttagaacaatgaactctcaaattactgctgcattatcaggtggtacaatctataacacagtttacactgctgatttctctcaaaattcagctgcaggtactgcagatatcatcagaagaattaattctggtttggctgcaaacccaaacgtttgttacatcttgcaaggttactcacaaggtgctgcagctacagttgttgctttgcaacaattaggtacttctggtgcagcttttaatgcagttaagggtgttttcttgatcggtaacccagatcataagtctggtttgacatgtaacgttgattcaaatggtggtactacaactagaaatgttaatggtttgtctgttgcttatcaaggttctgttccatcaggttgggtttcaaaaacattagatgtttgtgcttacggtgacggtgtttgtgatactgctcatggtttcggtattaatgcacaacatttgtcttatccatcagatcaaggtgttcaaactatgggttacaagttcgctgttaataagttgggtggttctgcataa

SEQ ID NO: 2 - protein, 239 aa, Cryptococcus sp. S-2
MLVSALALAVLSAASLGRAAPTPESAEAHELEARATSSACPQYVLINTRGTGEPQGQSAGFRTMNSQITAALSGGTIYNTVYTADFSQNSAAGTADIIRRINSGLAANPNVCYILQGYSQGAAATVVALQQLGTSGAAFNAVKGVFLIGNPDHKSGLTCNVDSNGGTTTRNVNGLSVAYQGSVPSGWVSKTLDVCAYGDGVCDTAHGFGINAQHLSYPSDQGVQTMGYKFAVNKLGGSA

SEQ ID NO: 3 - protein, 19 aa, Cryptococcus sp. S-2
MLVSALALAVLSAASLGRA

SEQ ID NO: 4 - DNA, 57 bp, synthetic construct
  misc_feature 1..57: note = Codon-optimised native secretion signal of CLE1
  source 1..57: mol_type = other DNA
atgttggtttcagcattggctttagcagttttgtctgctgcatcattaggtagagct

SEQ ID NO: 5 - protein, 33 aa, Trichoderma reesei
MVSFTSLLAGVAAISGVLAAPAAEVEPVAVEKR

SEQ ID NO: 6 - DNA, 99 bp, synthetic construct
  misc_feature 1..99: note = Codon-optimised secretion signal from Trichoderma reesei xyn2
  source 1..99: mol_type = other DNA
atggtctccttcacctccctcctcgccggcgtcgccgccatctcgggcgtcttggccgctcccgccgccgaggtcgaacccgtggctgtggagaagcgc

SEQ ID NO: 7 - DNA, 677 bp, synthetic construct
  misc_feature 1..677: note = Engineered TEF1i promoter
  source 1..677: mol_type = other DNA
gccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaagtatgtatctataaatttgaaaccaatattaggcggataagatgaaatagtgactggccaatccaggatttaatatgtcataaaagcctgttctaccttaatgggatgaatatccatgtctctgttatatgctccatagcacagatctagctacccatggtactttgagagaaggaaactacgtacttagtggtgatttacagttaaattcaatctacaattcaagtcattagcaaatagtctttatcccaaattctactagagttcggttttttactaacaagtatgtttttactttttactttatcatagaacatttaataaatc

SEQ ID NO: 8 - DNA, 720 bp, Cryptococcus sp. S-2
  source 1..720: mol_type = other DNA
atgctcgtctccgctctcgctctcgcggtgctgtccgctgcttctctcggccgagccgcaccaacgcccgagtccgccgaggcgcacgagctcgaggcccgcgccacgtccagcgcttgtccgcagtacgtcctgatcaacacgcgaggcacgggcgagccgcaaggccagtcggccggcttccgaacgatgaacagccagatcaccgccgcgctgtcgggtggcaccatctacaacactgtctacaccgccgatttcagccagaacagcgcggccggcacggccgacatcatccgccggatcaactcgggtctcgcggccaacccgaacgtgtgctacatcctccaagggtacagccagggcgcggctgctaccgtcgtcgcgctgcaacagctcggcacgagtggagcggcgttcaacgccgtcaagggtgtgttcctcattggcaacccggaccacaagtcgggcctgacttgcaacgtcgactcgaacggcggcactaccacacgcaatgtcaacggcctgtcggtcgcgtaccagggctcggtcccctcaggatgggtcagcaagactctcgatgtctgcgcttatggcgacggcgtgtgcgacaccgcgcacggattcggtatcaacgcacagcacctgtcgtaccctagtgaccaaggcgtccagaccatgggatacaagtttgccgtcaacaagcttggcgggtcggcctaa

SEQ ID NO: 9 - DNA, 977 bp, synthetic construct
  source 1..977: mol_type = other DNA
agtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgagtatgtatctataaatttgaaaccaatattaggcggataagatgaaatagtgactggccaatccaggatttaatatgtcataaaagcctgttctaccttaatgggatgaatatccatgtctctgttatatgctccatagcacagatctagctacccatggtactttgagagaaggaaactacgtacttagtggtgatttacagttaaattcaatctacaattcaagtcattagcaaatagtctttatcccaaattctactagagttcggttttttactaacaagtatgtttttactttttactttatcatagaacatttaataaatc

(19) United States
(12) Patent Application Publication
(10) Pub. No.: US 2025/0002965 A1
(43) Pub. Date: Jan. 2, 2025

(54) RECOMBINANT SACCHAROMYCES CEREVISIAE STRAINS FOR ENZYMATIC HYDROLYSIS OF BIOPLASTIC POLYMERS

(71) Applicants: Università degli Studi di Padova, Padova (IT); Stellenbosch University, Stellenbosch (ZA)

(72) Inventors: Lorenzo FAVARO, Padova (IT); Marinda Viljoen-Bloom, Stellenbosch (ZA); Marthinus Wessel Myburgh, Stellenbosch (ZA); Willem Heber van Zyl, Stellenbosch (ZA)

(21) Appl. No.: 18/701,026

(22) PCT Filed: Oct. 27, 2022
(86) PCT No.: PCT/IB2022/060342

§ 371 Date: Apr. 12, 2024

(30) Foreign Application Priority Data
Oct. 27, 2021 (GB) 2115470.3

(51) Int. Cl. C12P 21/02; C12N 1/18; C12N 9/18; C12N 15/81

(52) U.S. Cl. CPC C12P 21/02; C12N 1/18; C12N 9/18; C12N 15/81; C07K 2319/02; C12Y 301/01074

(57) ABSTRACT

The present invention relates to a method for producing a cutinase-like enzyme (CLE1) in a S. cerevisiae cell, comprising heterologously expressing a codon-optimised nucleic acid encoding the cutinase-like enzyme and operably linked to an engineered promoter in the cell. The invention further relates to recombinant S. cerevisiae cells capable of heterologously expressing the cutinase-like enzyme and to cutinase-like enzyme obtained from the cells or prepared by the methods. Also provided are methods of preparing a cell-free supernatant comprising a cutinase-like enzyme from the recombinant S. cerevisiae cells and the use thereof or of the recombinant S. cerevisiae cells in hydrolysing bioplastic polymers.

### BACKGROUND OF THE INVENTION

The present invention provides for methods of producing a cutinase-like enzyme (CLE) in a S. cerevisiae cell, comprising the heterologous expression of a codon-optimised nucleic acid encoding the cutinase-like enzyme under the control of an engineered yeast constitutive promoter in the cell. The invention further relates to recombinant S. cerevisiae cells capable of the heterologous expression of the cutinase-like enzyme. The cutinase-like enzyme produced by the methods or cells is useful in hydrolysing bioplastic polymers. Also provided are methods of preparing a cell-free supernatant comprising a cutinase-like enzyme from the recombinant S. cerevisiae cells and the use of the cell-free supernatant in hydrolysing bioplastic polymers.

Bioplastics is broadly defined as plastics that are produced from biomass or can degrade in natural/industrial environments or have both these properties. Although the bioplastics industry makes up a fraction of the global plastics industry, it continues to show steady growth in its share of the global plastic market and is set to be a $13.1 billion industry by 2027. Current waste management strategies for biodegradable bioplastics such as starch blends and polylactic acid (PLA), which are two of the main drivers in the bioplastic industry, form part of traditional organic waste treatment facilities. PLA is one of the most popular biodegradable bioplastics due to its high mechanical strength, high modulus, biodegradability, biocompatibility, bioabsorbability, transparency, energy savings, low toxicity and processability. Its monomer (lactic acid) is produced from plant biomass through microbial fermentation; the lactic acid is then polymerised to form PLA. Anaerobic digestion and composting are currently the preferred end-of-life treatments for these materials and will continue to fulfill this role in the foreseeable future. Although PLA is considered as biodegradable, recent studies showed that several bioplastics persist throughout treatment, resulting in reduced product value and processing constraints. Furthermore, life cycle analyses have confirmed that new and efficient recycling systems should be a priority for the long-term sustainable use of these materials.

Microbial hydrolases capable of bioplastics degradation have previously been identified and their use for the large-scale treatment of bioplastics may have several advantages over current systems. Hydrolytic enzymes can speed-up traditional processes for the treatment of bioplastics, reduce the risk of compromising composting and anaerobic digestion systems and can remove these materials from the final product. Moreover, these enzymes can function at mild temperatures and pH, have specificity towards specific polymers and could deliver pure monomers that paves the way for a cradle-to-cradle recycling system for bioplastics. However, there are currently no commercial enzyme preparations specifically designed for the large-scale treatment of bioplastics. Several factors still need to be addressed for enzyme-based strategies to be a viable option for the large-scale treatment of bioplastics, either as sole treatment or in combination with existing strategies. First, a cost-effective and scalable enzyme production process is required to produce cocktails capable of efficient bioplastic degradation. Second, enzyme-based treatment processes need to be simplified to function efficiently at mild temperatures, pH levels and without the need for additional pretreatment of materials or the use of solvents, emulsifiers and other chemical catalysts.

Research papers describing the use of extracellular enzymes for the treatment of bioplastics have focused on conventional prokaryotic hosts, such as Escherichia coli, in recombinant enzyme production systems or make use of non-conventional prokaryotic and eukaryotic hosts to produce native extracellular enzymes. The use of conventional eukaryotic hosts to produce recombinant enzymes has several advantages over prokaryotic and non-conventional eukaryotic systems. This includes well-established genetic manipulation protocols, improved enzyme production titers, complex protein production and secretion machinery, ease of protein purification, GRAS status, growth on cheap substrates and the use of existing industrial processes and infrastructure for their large-scale cultivation.

Fungi are known to produce a myriad of hydrolytic enzymes. Indeed, several fungal hydrolases capable of plastic degradation have been characterized, but their use is still relatively underexplored, especially compared to bacterial enzymes. Cutinase-like enzymes (CLEs) are of particular interest as they have been shown to hydrolyse a broad spectrum of bioplastics. These enzymes are typically associated with plant pathogens including several fungal species. It follows that industrially applicable and conventional fungal hosts are especially adept at producing extracellular enzymes originating from other eukaryotic organisms, including other fungi. Still, there are major hurdles that need to be overcome when over-expressing genes from native eukaryotes in other eukaryotic organisms traditionally used in industry, with expression levels of native genes typically being low. Major bottlenecks in heterologous expression of native fungal genes include, among others, sub-optimal GC content, low codon bias indexes, presence of tandem rare codons, the lack of strong promoters and efficient secretion to drive enzyme production. Thus, the inventors of the present invention have used molecular techniques and genetic engineering approaches in industrially applicable fungal hosts for the improved production of bioplastic-degrading enzymes, including CLEs, for application during the treatment of bioplastic polymers.

Specifically, the inventors of the present invention have constructed genetically modified industrially applicable eukaryotic hosts that show enhanced expression of a CLE1 gene from Cryptococcus sp. S-2 and increased hydrolysis of bioplastics.

### SUMMARY OF THE INVENTION

The invention relates to a method for producing a cutinase-like enzyme (CLE1) in a S. cerevisiae cell, wherein the method comprises heterologously expressing a codon-optimised nucleic acid encoding the cutinase-like enzyme and operably linked to an engineered promoter in the cell. The invention also relates to recombinant S. cerevisiae cells capable of heterologously expressing the cutinase-like enzyme and to a cutinase-like enzyme obtained from the cells or prepared by the methods described. The invention further relates to methods of preparing a cell-free supernatant comprising the cutinase-like enzyme from the recombinant S. cerevisiae cells and the use of the cutinase-like enzyme in hydrolysing bioplastic polymers.

According to a first aspect of the invention there is provided for a method for producing a cutinase-like enzyme (CLE1) in a S. cerevisiae cell, the method comprising and/or consisting of: heterologously expressing a nucleic acid encoding the CLE1 (the CLE1 gene) in the cell, wherein the nucleic acid encoding the CLE1 is codon-optimised for expression in S. cerevisiae, further wherein the nucleic acid encoding the CLE1 is operably linked to an engineered promoter.

In a first embodiment of the method, the cutinase-like enzyme may have the amino acid sequence of SEQ ID NO:2.

According to a second embodiment of the method, the nucleic acid encoding the CLE1 has a nucleotide sequence having at least about 80%, at least about 90%, about 95%, about 96%, about 97%, about 98% or about 99% sequence identity to SEQ ID NO: 1. In one embodiment of the method, the nucleic acid encoding the CLE1 has a nucleotide sequence substantially identical to SEQ ID NO:1.

In a third embodiment of the method of the invention, the engineered promoter may be a TDHi engineered promoter having a nucleotide sequence having at least about 80%, at least about 90%, about 95%, about 96%, about 97%, about 98% or about 99% sequence identity to SEQ ID NO:9, or substantially identical to SEQ ID NO:9 or a TEF1i engineered promoter having a nucleotide sequence having at least about 80%, at least about 90%, about 95%, about 96%, about 97%, about 98% or about 99% sequence identity to SEQ ID NO:7, or substantially identical to SEQ ID NO:7. It will be appreciated by those of skill in the art that other engineered promoters may be suitable for producing the CLE1 enzyme from the CLE1 gene, including, but not limited to, a promoter selected from the group consisting of the ENO1i, ADH2i, TDH3i, HXT7i, ENO1cxi, TDH3cxi, HXT7cxi and TEF1cxi engineered promoters.

According to a fourth embodiment of the method of the invention, the CLE1 enzyme may comprise a secretion signal. Suitably, the secretion signal may have the amino acid sequence of SEQ ID NO:3 or SEQ ID NO:5.

In a further embodiment of the method of the invention, the S. cerevisiae cell may be of the strain S. cerevisiae Y294, S. cerevisiae Ethanol Red V1, S. cerevisiae M2n, or S. cerevisiae Y130.

In one embodiment of the method of the invention, the method may further comprise culturing the S. cerevisiae cell to obtain a population of S. cerevisiae cells.

In yet another embodiment of the method of producing a CLE1 enzyme of the invention, the method may additionally comprise preparing a cell-free supernatant from the population of S. cerevisiae cells, wherein the cell-free supernatant comprises the CLE1 enzyme.

According to a second aspect of the present invention, there is provided for a recombinant S. cerevisiae cell comprising: a nucleic acid encoding a cutinase-like enzyme (CLE1), wherein the nucleic acid encoding the CLE1 is codon-optimised for expression in S. cerevisiae, further wherein the nucleic acid encoding the CLE1 is operably linked to an engineered promoter, and wherein the recombinant S. cerevisiae cell is capable of heterologously expressing the CLE1.

In a first embodiment of the recombinant S. cerevisiae cell, the cutinase-like enzyme may have the amino acid sequence of SEQ ID NO:2.

According to a second embodiment of the recombinant S. cerevisiae cell of the invention, the nucleic acid encoding the CLE1 has a nucleotide sequence having at least about 80%, at least about 90%, about 95%, about 96%, about 97%, about 98% or about 99% sequence identity to SEQ ID NO:1. In one embodiment of the recombinant S. cerevisiae cell, the nucleic acid encoding the CLE1 has a nucleotide sequence substantially identical to SEQ ID NO:1.

In a third embodiment of the recombinant S. cerevisiae cell of the invention, the engineered promoter may be a TDHi engineered promoter having a nucleotide sequence having at least about 80%, at least about 90%, about 95%, about 96%, about 97%, about 98% or about 99% sequence identity to SEQ ID NO:9, or substantially identical to SEQ ID NO:9 or a TEF1i engineered promoter having a nucleotide sequence having at least about 80%, at least about 90%, about 95%, about 96%, about 97%, about 98% or about 99% sequence identity to SEQ ID NO:7, or substantially identical to SEQ ID NO:7. It will be appreciated by those of skill in the art that other engineered promoters may be suitable for producing the CLE1 enzyme from the CLE1 gene, including, but not limited to, a promoter selected from the group consisting of the ENO1i, ADH2i, TDH3i, HXT7i, ENO1cxi, TDH3cxi, HXT7cxi and TEF1cxi engineered promoters.

According to a fourth embodiment of the recombinant S. cerevisiae cell of the invention, the CLE1 enzyme may comprise a secretion signal. Suitably, the secretion signal may have the amino acid sequence of SEQ ID NO:3 or SEQ ID NO:5.

In a further embodiment of the recombinant S. cerevisiae cell of the invention, said S. cerevisiae cell may be of the strain S. cerevisiae Y294, S. cerevisiae Ethanol Red V1, S. cerevisiae M2n, or S. cerevisiae Y130.

According to a third aspect of the present invention, there is provided for a cutinase-like enzyme produced by the method of the invention described herein or heterologously produced by the recombinant S. cerevisiae cell of the invention described herein. In one embodiment, the cutinase-like enzyme may have the amino acid sequence of SEQ ID NO:2

A fourth aspect of the present invention provides for a method of preparing a cell-free supernatant comprising a cutinase-like enzyme, said method comprising: culturing the recombinant S. cerevisiae cell of the invention described herein to obtain a population of S. cerevisiae cells; and preparing a cell-free supernatant from the population of S. cerevisiae cells.

In one embodiment of the method of preparing a cell-free supernatant, the method may further comprise concentrating the cell-free supernatant. Suitable methods for concentrating the cell-free supernatant include, but are not limited to, concentrating the cell-free supernatant by lyophilisation, filtration and/or chromatography.

According to a fifth aspect of the present invention there is provided for a cell-free supernatant prepared by the methods described herein.

In another aspect of the invention, use of a cutinase-like enzyme produced by the method of the invention described herein or heterologously produced by the recombinant S. cerevisiae cell of the invention described herein, or use of the cell-free supernatant prepared by the methods described herein is contemplated for hydrolysing a bioplastic polymer.

Methods of hydrolysing a bioplastic polymer, comprising incubating the polymer with a cutinase-like enzyme produced by the method of the invention described herein or heterologously produced by the recombinant S. cerevisiae cell of the invention described herein or with the cell-free supernatant prepared by the methods described herein, also form the subject of the present invention. Suitable bioplastics to be hydrolysed by the cell-free supernatant prepared by the methods described herein may include, without limitation and in any combination or mixture thereof, polylactic acid (PLA), including Poly(L-lactide) (PLLA), Poly(D-lactide) (PDLA), and Poly(DL-lactide) (PDLLA), Polycaprolactone (PCL), Polybutylene succinate (PBS), Polybutylene adipate terephthalate (PBAT), Polyhydroxyalkanoates (PHAs) and Thermoplastic starch (TPS) blends.

According to a further embodiment of the method of hydrolysing a bioplastic polymer, incubating the polymer with the cutinase-like enzyme or the cell-free supernatant may take place between about 42° C. and about 45° C., including at about 42° C. or about 45° C. and at a pH of about 6.8-7.

In a further aspect of the present invention there is provided for a method of hydrolysing a bioplastic polymer, comprising incubating the bioplastic polymer with a recombinant S. cerevisiae cell of the invention or a S. cerevisiae cell expressing a cutinase-like enzyme of the invention. In one embodiment, the recombinant S. cerevisiae cell may be a strain variant that is not capable of consuming hydrolysis products of the method of hydrolysing a bioplastic polymer.

Also contemplated according to the invention is a bioreactor comprising the recombinant S. cerevisiae cell of the invention described herein, wherein the bioreactor is configured for heterologous production of a cutinase-like enzyme by the recombinant S. cerevisiae cell and subsequent hydrolysis of a bioplastic polymer by the cutinase-like enzyme.

### BRIEF DESCRIPTION OF THE FIGURES

Non-limiting embodiments of the invention will now be described by way of example only and with reference to the following figures:

FIG. 1: Plasmid maps of S. cerevisiae expression vectors described herein. A=Plasmid pBBH4 is used herein as a control and does not include a gene encoding the CLE1 enzyme; B=The pBBH4-CLExs plasmid was constructed to include a codon-optimised gene (SEQ ID NO: 1) encoding cutinase-like enzyme from Cryptococcus sp. S-2 with XYNSEC (SEQ ID NO:5) secretion signal; C=The pBBH4-CLEns plasmid was constructed to include a codon-optimised gene encoding cutinase-like enzyme from Cryptococcus sp. S-2 with a native secretion signal (SEQ ID NO:3) and the engineered TEF1i promoter (SEQ ID NO:7) responsible for expression; D=Plasmid pBBH4-CLEwt plasmid was constructed to include the native cutinase-like enzyme gene from Cryptococcus sp. S-2 (SEQ ID NO:8) with its native secretion signal (SEQ ID NO:3) and the engineered TEF1i promoter (SEQ ID NO:7) responsible for expression; E=The pBBH4-CLEns-TDHi plasmid was constructed to include a codon-optimised gene encoding cutinase-like enzyme from Cryptococcus sp. S-2 (SEQ ID NO:1) with its native secretion signal (SEQ ID NO:3) and the engineered TDHi promoter (SEQ ID NO:9) responsible for expression.

FIG. 2: SDS-PAGE gel showing the presence of the CLE1 protein species (black arrows) in the supernatant of the Y294 [CLExs] and Y294 [CLEns] strains while the protein species is absent in the supernatant of the Y294 [BBH] strain. Molecular marker is indicated on the far left.

FIG. 3: Photograph showing hydrolysis halo formation by Y294 [CLExs], Y294 [CLEns] and Y294 [BBH] strains on tributyrin containing agar plates. A larger hydrolysis halo is observed for the Y294 [CLEns] strain compared to Y294 [CLExs] and no halo is observed around the Y294 [BBH] control.

FIG. 4: A turbidity-based assay was developed to evaluate whether the secreted CLE1 protein species was active in hydrolysing emulsified PLA substrates (Solid lines=PLLA; Dashed lines=PDLA, representing the polymers from L- and D-lactic acid isomers, respectively). Proteinase-K (0.5 mg/ml in 0.1M KH₂PO₄ buffer), hereafter referred to as commercial ProK (●), was used as positive control during assays on PLLA. The Y294 [BBH] (♦) Y294 [CLEns] (▪) and Y294 [CLExs] (▴) yeast strains were cultivated in 2×SC-URA media and activity was determined after 24, 48 and 72 h of growth. Error bars represent standard deviation from the mean of three replicates. The ProK positive control delivered constant activity over multiple assay rounds on the PLLA substrate while the Y294 [BBH] control showed no significant activity on either substrates. Both recombinant yeast strains showed an increase in activity over time on both PLA substrates.

FIG. 5: Graphs showing the percentage hydrolysis of PDLA (dotted bars) and PLLA (striped bars) emulsions after 72 h hydrolysis using cell-free supernatant from the various yeast strains and commercial ProK. Error bars represent standard deviation from the mean of three replicates. Complete hydrolysis of the PDLA emulsion was reached after 72 h hydrolysis by the recombinant Y294[CLEns] and Y294[CLExs] strains while the Y294[BBH] control and commercial ProK did not show any significant decrease in turbidity. Near-complete hydrolysis of the PLLA emulsion was also observed after 72 h hydrolysis for both the recombinant Y294[CLEns] and Y294[CLExs] strains and the commercial ProK positive control.

FIG. 6: Results from HPLC analysis indicating lactic acid production from various substrate loadings of PLLA (solid lines) and PDLA (dashed lines) powders. A=4 g/L; B=10 g/L and C=25 g/L PLA powder substrate loadings. Error bars represent standard deviation from the mean of three replicates. It is evident that lactic acid production titre increases as substrate concentration increases. The Y294[CLEns] strain outperformed its Y294[CLExs] counterpart on both substrates.

FIG. 7: Lactic acid production ratios and productivity during cell-free hydrolysis of PLA powders. Cn represents the lactic acid concentration at each time point, while Cf is the final concentration of lactic acid produced in each setup. The ratio of lactic acid produced at each time point relative to the final concentration (%) as well as lactic acid productivity (g/L/h) is given. Shading indicates the performance of the strains at each time point (darker shading=better performance). NA=Not available.

FIG. 8: Results from HPLC analysis indicating lactic acid production from PLLA (solid lines) and PDLA (dashed and dotted lines) films over 10 day hydrolysis period at 37° C. Error bars represent standard deviation from the mean of three replicates. Substantial amounts of lactic acid are produced by hydrolysing PLA films with supernatant from recombinant Y294[CLExs] and Y294[CLEns] strains.

FIG. 9: Lactic acid production ratios and productivity during cell-free hydrolysis of PLA films. Cn represents the lactic acid concentration at each time point, while Cf is the final concentration of lactic acid produced in each setup. The ratio of lactic acid produced at each time point relative to the final concentration (%) as well as lactic acid productivity (g/L/h) is given. Shading indicates the performance of the strains at each time point (darker shading=better performance).

FIG. 10: SEM images of PDLA film surface during hydrolysis trials. A=PDLA film prior to treatment with cell-free supernatant; B=PDLA film surface incubated for 168 h with cell-free supernatant from Y294[BBH] cells; C=PDLA film surface incubated for 168 h with cell-free supernatant from Y294[CLEns] cells. Clear hydrolysis patterns are observed on film incubated with supernatant from the recombinant Y294 [CLEns] strain while no signs of hydrolysis were observed for the Y294[BBH4] strain.

FIG. 11: SEM images of PDLA film surface during hydrolysis trials. A=PDLA film surface incubated for 24 h with cell-free supernatant from Y294[CLEns] cells; B=PDLA film surface incubated for 48 h with cell-free supernatant from Y294[CLEns] cells; white arrow indicates the hydrolysed amorphous region while black arrow shows an unhydrolysed crystalline area.

FIG. 12: SEM images of PLLA film surface during hydrolysis trials. A=PLLA film surface incubated for 24 h with cell-free supernatant from Y294 [CLEns] cells; white arrows indicate amorphous regions with clear pit formation while black arrows show unhydrolysed crystalline areas; B=PLLA film surface incubated for 24 h with commercial ProK (0.5 mg/mL in 0.1M KH₂PO₄ buffer) showing a more general attack on the film surface.

FIG. 13: SEM images of PLLA film surface during hydrolysis trials. A=PLLA film surface incubated for 24 h with cell-free supernatant from Y294[CLEns] cells; B=PLLA film surface incubated for 24 h with commercial ProK (0.5 mg/mL in 0.1M KH₂PO₄ buffer).

FIG. 14: Graph showing PLLA film crystallinity during hydrolysis with cell-free supernatant from Y294[CLEns] (▪) and Y294[BBH] (♦) strains based on differential scanning calorimetry analysis. A sharp increase followed by substantial decrease in crystallinity confirms the hydrolysis of amorphous regions prior to more crystalline areas by the recombinant strain.

FIG. 15: Graphs showing data from small scale hydrolysis trials on PLA films after 240 h incubation. A=Percentage weight loss relative to the Y294[BBH] control; B=lactic acid concentration determined through HPLC analysis. Error bars represent standard deviation from the mean of three replicates. Substantial weight loss and lactic acid production is observed at the end of hydrolysis with cell-free supernatant from the recombinant yeast strains.

FIG. 16: Photographs showing the effect of using cell-free supernatant from the Y294[BBH] and Y294[CLEns] strains for 240 h scaled-up hydrolysis of various PLA films. PDLA_{HMW} and PLLA_{HMW} refer to higher molecular weight PLA materials. Extreme fragmentation is observed for films incubated with supernatant from the Y294[CLEns] strain.

FIG. 17: Graphs showing data from scaled-up hydrolysis trials on various PLA films after 240 h incubation. A=Percentage weight loss relative to the Y294[BBH] control; B=lactic acid concentration determined through HPLC analysis. Error bars represent standard deviation from the mean of three replicates. Similar weight loss and lactic acid concentrations to small-scale hydrolysis trials are observed after scaling up the reaction volumes.

FIG. 18: SDS-PAGE gel showing the presence of the CLE1 protein species (black brackets) in the supernatant of the Y294[CLEns]-TDHi and Y294[CLEns] strains while the protein species is absent in the supernatant of the Y294[BBH] strain. Molecular marker is indicated on the far left. It is evident that the Y294[CLEns]-TDHi strain delivered substantially more CLE1 in the extracellular fraction than the previously constructed Y294[CLEns] strain.

FIG. 19: Photograph showing hydrolysis halo formation by Y294[CLEns]-TDHi, Y294[CLEns] and Y294[BBH] strains on 0.035 g/L Polycaprolactone containing agar plates. A larger hydrolysis halo is observed for the Y294[CLEns]-TDHi strain compared to Y294[CLEns] and no halo is observed around the Y294[BBH] control.

FIG. 20: A turbidity-based assay was used to evaluate the extracellular PDLA hydrolytic activity of CLE1 producing S. cerevisiae strains under control of different engineered promoters, including the codon-optimised CLE1 gene and the wildtype gene. The Y294[BBH] (▴) Y294[CLEwt] (x), Y294[CLEns]-TDHi (●) and Y294[CLEns] (▪) yeast strains were cultivated in 2×SC-URA media and extracellular PLA hydrolytic activity was determined after 24, 48 and 72 h of growth. Error bars represent standard deviation from the mean of three replicates. The Y294[CLEwt] strain delivered 1.4 times lower activity than Y294[CLEns] while the Y294[CLEns]-TDHi showed a 1.5-fold increase compared to Y294[CLEns].

FIG. 21: The optimal temperature for PLA hydrolysis using recombinant CLE1 was determined using a turbidity-based enzyme activity assay conducted at various incubation temperatures. The Y294[BBH] (▴) and Y294[CLEns]-TDHi (●) strains were cultivated in 2×SC-URA media for 72 h after which supernatant was collected through centrifugation and used in the assays at different temperatures. Extracellular PLA hydrolytic activity from the Y294[CLEns]-TDHi strain was 1.6-fold higher at 42° C. (69 U/mL) than at the previously reported optimal temperature of 37° C. (44 U/mL).

FIG. 22: The stability of recombinant CLE1 in the supernatant of the Y294[CLEns]-TDHi strain was determined by incubating the supernatant at 37, 42, 45 and 50° C. for a total of 120 h. Residual activity at each of the temperatures was determined using a turbidity-based enzyme assay after 72 h and 120 h of incubation. Recombinant CLE1 in the supernatant was very stable at 37 and 42° C. after 120 h incubation with 100 and 94% residual activity detected after incubation at the two respective temperatures. A significant decrease in residual activity was detected after 120 h incubation at 45° C. (75% residual activity) as well as 50° C. (28% residual activity).

FIG. 23: Results from HPLC analysis indicating lactic acid released from 10 g/L PLLA films over 10-day hydrolysis period at 37° C. in small scale hydrolysis trials using supernatant from the Y294[CLEns] (▪), Y294[CLEns]-TDHi (●) and Y294[BBH] (▴) strains. Error bars represent standard deviation from the mean of three replicates. Substantial amounts of lactic acid are released by hydrolysing PLA films with supernatant from recombinant Y294[CLEns] and Y294[CLEns]-TDHi strains. After 24 h hydrolysis, supernatant from the Y294[CLEns]-TDHi strain released almost 3 g/L more lactic acid than when supernatant from the Y294[CLEns] strain was used.

FIG. 24: Results from HPLC analysis indicating lactic acid released from 10 g/L PLLA films over 7-day hydrolysis period in Bioreactor experiments using 700 mL supernatant from the Y294[CLEns]-TDHi strain. Supernatant was collected from the Y294[CLEns]-TDHi strain through centrifugation after 72 h cultivation. Three hydrolysis set-ups were investigated for improved PLA hydrolysis, the first conducted at 37° C. and non-neutralising conditions (no pH control) (▴) the second at 37° C. and neutralising conditions (pH maintained at 6.8-7 through addition of 3M KOH) (●) and the third at 42° C. and neutralising conditions (pH maintained at 6.8-7 through addition of 3M KOH) (▪). Error bars represent standard deviation from the mean of three replicates. The effect of neutralising conditions is evident in the release of higher lactic acid concentrations compared to non-neutralising conditions at 37° C. The higher PLA hydrolytic activity of CLE1 at 42° C. vs 37° C. resulted in the release of 11.7 g/L lactic acid after 48 h which is 4.5 g/L higher than that found at 37° C. Maximum lactic acid concentration released reached 14.9 g/L after 7-days hydrolysis at 42° C. and neutralising conditions which resulted in a 78% weight loss of PLA films.

### SEQUENCE LISTING

The nucleic acid and amino acid sequences listed in the accompanying sequence listing are shown using standard letter abbreviations for nucleotide bases and the standard three-letter abbreviations for amino acids. It will be understood by those of skill in the art that only one strand of each nucleic acid sequence is shown, but that the complementary strand is included by any reference to the displayed strand. The accompanying sequence listing is hereby incorporated by reference in its entirety. In the accompanying sequence listing:
SEQ ID NO:1—nucleotide sequence of codon-optimised CLE1 gene.
SEQ ID NO:2—amino acid sequence of CLE1.
SEQ ID NO:3—amino acid sequence of native secretion signal of CLE1.
SEQ ID NO:4—nucleotide sequence encoding codon-optimised native secretion signal of CLE1.
SEQ ID NO:5—amino acid sequence of secretion signal from Trichoderma reesei xyn2.
SEQ ID NO:6—nucleotide sequence encoding codon-optimised secretion signal from Trichoderma reesei xyn2.
SEQ ID NO:7—nucleotide sequence of TEF1i promoter.
SEQ ID NO:8—nucleotide sequence of native (not codon-optimised) CLE1 gene.
SEQ ID NO:9—nucleotide sequence of TDHi promoter.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims that follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

The present invention relates in its broadest sense to the use of molecular engineering techniques such as codon-optimisation and engineered yeast promoters to construct a recombinant eukaryotic host that shows enhanced heterologous expression of a cutinase-like enzyme (CLE), resulting in improved recombinant enzyme production. It also describes the use of culture supernatant from the recombinant yeast strains in processes involving the treatment of bioplastics, such as polylactic acid (PLA), in various forms and concentrations. Quantitative data from PLA hydrolysis is provided as well as insights into the degradation mechanism of the CLE1 enzyme on PLA films.

The inventors of the present invention have constructed genetically modified, industrially applicable, eukaryotic hosts that show enhanced expression of a CLE1 gene from Cryptococcus sp. S-2. Following an in-silico evaluation of the expressive potential of the CLE1 gene in the S. cerevisiae host, the inventors identified key points where heterologous expression could be improved and were able to significantly enhance CLE1 expression and recombinant production in S. cerevisiae with concomitant improvements in PLA hydrolysis using the recombinant S. cerevisiae. The use of crude supernatant, without purification or concentration, delivered improved PLA hydrolysis compared to previously reported data while substantial corroborating results are also supplied herein. Therefore, this work shows a potentially significant step towards commercial CLE1 production by recombinant eukaryotic strains for the degradation of bioplastic materials, such as commercially available PLA and starch blends, in a simple yet efficient treatment process.

In one embodiment of the present invention, there is provided for a method for constructing recombinant S. cerevisiae strains to overexpress a bioplastic degrading enzyme at exceptionally high titers. The invention entails the use of molecular techniques such as codon-optimisation together with the use of an expression enhancing engineered yeast promoter, for example a TEF1i (Myburgh et al., 2020) (SEQ ID NO:7) or TDHi promoter (SEQ ID NO:9), to deliver improved expression and production of the CLE1 enzyme in a yeast host cell. Here the inventors show that codon-optimisation significantly improves extracellular protein production, resulting in higher hydrolytic activity on the PDLA emulsion in the turbidity-based enzyme assays. In particular, the inventors of the present invention show that strains comprising the engineered TDHi promoter produced significantly more CLE1 in the extracellular fraction than the TEF1i promoter.

In another embodiment of the present invention, there is provided for the use of the extracellular fraction of the developed recombinant strains for the degradation of bioplastics, which allows for significant fragmentation, weight loss and monomer production from commercial PLA materials. In one embodiment, the method for the treatment of PLA includes the use of a recombinant yeast strain that produces CLE1 in its extracellular fraction that can be used as-is at mild temperatures and PH levels without any material pretreatment, the addition of solvents, activity enhancing emulsifiers and/or other catalysts.

The data presented herein provides the first quantitative analysis of PLA hydrolysis using a recombinant yeast strain having CLE1 activity. In one embodiment, the CLE1 enzyme is encoded by a codon-optimised gene under the control of an engineered yeast promoter to significantly improve heterologous CLE1 expression in S. cerevisiae that effects the recombinant strain to produce the bioplastic degrading CLE1 enzyme at exceptionally high titers in the extracellular fraction. The effect of codon-optimisation on CLE1 expression potential in S. cerevisiae is demonstrated through an in-silico analysis of the native and codon-optimised genes (Table 1) as well as the experimental data presented. In another embodiment, the recombinant strains of the present invention are demonstrated to exhibit improved hydrolysis capacity on different types of PLA materials (PLLA and PDLA) through hydrolysis trials on various substrate loadings of PLA emulsions, powders, and films by quantifying turbidity, lactic acid production, film weight loss and fragmentation. Most notably, the hydrolysis of various PLA substrates is achieved without any purification or concentration of culture supernatant, at mild temperatures and PH levels, while mitigating the use of any additional pretreatments, solvents, emulsifiers, or other catalysts. Thus, the process by which the PLA materials are degraded using the recombinant strains of the present invention is demonstrably simplified compared to processes using known CLE1-expressing strains. Furthermore, trials using PLA emulsions show the complete hydrolysis of higher loadings of PLA compared to the known CLE1-producing strains in a similar hydrolysis time. In addition, scanning electron micrographs and differential scanning calorimetry on hydrolyzed PLA films give insights into the degradation mechanism of the CLE1 enzyme on PLA films, which differs from Proteinase K, herein after referred to as commercial ProK, a commercial enzyme typically used for PLA hydrolysis.

In a further embodiment, optimised processing parameters are provided for the hydrolysis of PLA substrates using the cell-free supernatant obtained from the recombinant S. cerevisiae strains. In particular, the recombinant codon-optimised CLE1 in the crude supernatant showed a 1.6-fold increase in activity on emulsified PDLA at 42° C. compared to the activity at 37° C. The enzyme in the crude supernatant was confirmed to be stable at the higher temperature, retaining 94% activity after 120 h at 42° C., 75% at 45° C. and 28% at 50° C. Thus, the optimal temperature for the hydrolysis of the PLA substrates with the codon-optimised CLE1 of 42° C. is higher than the 37° C. previously reported for the wild-type enzyme. In addition, the inventors showed that maintaining pH at 6.8-7.0 does not improve lactic acid release within the first 48 h, however, the benefit of pH control from 72 h onwards is evident, with lactic acid reaching 12.7 g/L after 168 h, correlating to a 1.4-fold increase compared to non-neutralising conditions at 37° C. Conducting the process under neutralising conditions and at the newly identified optimum temperature for PLA hydrolysis (42° C.) significantly increased lactic acid release within the first 48 h (11.7 g/L released), which was 1.6-fold higher than at 37° C., correlating to a productivity of 0.25 g/L/h. The final lactic acid concentration after 168 h of hydrolysis at 42° C. with pH control was 14.92 g/L as opposed to 9.36 g/L at 37° C. without pH control.

The term “bioplastic polymer” refers to any material which is either biodegradable or non-biodegradable and is derived from bio-based or fossil fuel-based resources. These include but are not limited to Polylactic acid (PLA), including Poly(L-lactide) (PLLA), Poly(D-lactide) (PDLA), and Poly(DL-lactide) (PDLLA), Polycaprolactone (PCL), Polybutylene succinate (PBS), Polybutylene adipate terephthalate (PBAT), Polyhydroxyalkanoates (PHAs), Thermoplastic starch (TPS) blends and Polyethylene terephthalate (bio-PET).

A “protein,” “peptide” or “polypeptide” is any chain of two or more amino acids, including naturally occurring or non-naturally occurring amino acids or amino acid analogues, irrespective of post-translational modification (e.g., glycosylation or phosphorylation).

The terms “nucleic acid”, “nucleic acid molecule” and “polynucleotide” are used herein interchangeably and encompass both ribonucleotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides.

The term “isolated”, is used herein and means having been removed from its natural environment.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids and carbohydrates which it is ordinarily associated with in its natural state.

The term “cell-free supernatant” as used herein refers to culture broth from which yeast cells have been removed, but that contains all extracellular products including recombinant proteins and is predominantly collected at the end of a certain culturing period. Cell-free supernatant typically refers to the broth in its unconcentrated form where the products it contains have not been purified through dialysis, chromatography, filtration or any other methods. Cell-free supernatant can be collected through centrifugation (typically 5 min at 4000 rpm for yeast) and/or filtration of cultures.

The term “complementary” refers to two nucleic acids molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the activity of one or more of the expressed polypeptides or of the polypeptides encoded by the nucleic acid molecules. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 h at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Those skilled in the art will appreciate that polypeptides, peptides or peptide analogues, including the CLE1 enzymes of the present invention, can be prepared from their corresponding nucleic acid molecules using recombinant DNA technology. Polypeptides, peptides and peptide analogues can also be synthesised using standard chemical techniques, for instance, by automated synthesis using solution or solid phase synthesis methodology. Automated peptide synthesisers are commercially available and use techniques known in the art.

As used herein, the term “gene” refers to a nucleic acid that encodes a functional product, for instance an RNA, polypeptide or protein. A gene may include regulatory sequences upstream or downstream of the sequence encoding the functional product.

As used herein, the term “coding sequence” refers to a nucleic acid sequence that encodes a specific amino acid sequence. On the other hand, a “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, and translation leader sequences.

In some embodiments, the genes used in the method of the invention may be operably linked to other sequences. By “operably linked” is meant that the nucleic acid molecules encoding the cutinase-like enzymes of the invention and regulatory sequences are connected in such a way as to permit expression of the proteins when the appropriate molecules are bound to the regulatory sequences. Such operably linked sequences may be contained in vectors or expression constructs which can be transformed or transfected into host cells for expression. It will be appreciated that any vector or vectors can be used for the purposes of expressing the cutinase-like enzymes of the invention.

The term “promoter” refers to a DNA sequence that is capable of controlling the expression of a nucleic acid coding sequence or functional RNA. A promoter may be based entirely on a native gene promoter, or it may be comprised of different elements from different promoters found in nature. Different promoters are capable of directing the expression of a gene in different cell types, or at different stages of development, or in response to different environmental or physiological conditions. A “constitutive promoter” is a promoter that direct the expression of a gene of interest in most host cell types most of the time. An “engineered promoter” refers to a promoter that does not naturally occur in the form it is used for the recombinant expression of heterologous genes. The engineered promoter can be made up of various regulatory elements such as 3′-UAS, 5′-UTR introns and native promoters to improve the expression of heterologous genes by the promoter.

The term “recombinant” means that something has been recombined. When used with reference to a nucleic acid construct the term refers to a molecule that comprises nucleic acid sequences that are joined together or produced by means of molecular biological techniques. The term “recombinant” when used in reference to a protein or a polypeptide refers to a protein or polypeptide molecule which is expressed from a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e. by human intervention. Recombinant nucleic acid constructs may be introduced into a host cell by transformation. Such recombinant nucleic acid constructs may include sequences derived from the same host cell species or from different host cell species.

The term “vector” refers to a means by which polynucleotides or gene sequences can be introduced into a cell. There are various types of vectors known in the art including plasmids, viruses, bacteriophages and cosmids. Generally, polynucleotides or gene sequences are introduced into a vector by means of a cassette. The term “cassette” refers to a polynucleotide or gene sequence that is expressed from a vector, for example, the polynucleotide or gene sequences encoding the cutinase-like enzymes of the invention. A cassette generally comprises a gene sequence inserted into a vector, which in some embodiments, provides regulatory sequences for expressing the polynucleotide or gene sequences. In other embodiments, the vector provides the regulatory sequences for the expression of the cutinase-like enzymes. In further embodiments, the vector provides some regulatory sequences, and the nucleotide or gene sequence provides other regulatory sequences. “Regulatory sequences” include but are not limited to promoters, transcription termination sequences, enhancers, splice acceptors, donor sequences, introns, ribosome binding sequences, poly (A) addition sequences, and/or origins of replication.

The term “heterologous expression” or “heterologously expressing” refers to the expression of a gene or part of a gene in a host organism that does not naturally have this gene or gene fragment. Insertion of the gene in the heterologous host is performed by recombinant technology. In one non-limiting example, a cutinase-like enzyme from Cryptococcus sp. S-2 may be heterologously expressed in a non-native host cell, such as a S. cerevisiae cell, including but not limited to S. cerevisiae Y294, S. cerevisiae Ethanol Red V1, S. cerevisiae M2n or S. cerevisiae YI30.

In some embodiments, the recombinant yeast strains expressing cutinase-like enzymes, the cell-free supernatant of such strains, or the cutinase-like enzymes of the present invention are well-suited for hydrolysis of different bioplastics, bioplastic mixtures and biocompounds made of bioplastics. Such bioplastics may include, without limitation and in any combination or mixture of, polylactic acids (PLA), including Poly(L-lactide) (PLLA), Poly(D-lactide) (PDLA), and Poly(DL-lactide) (PDLLA), as well as Polycaprolactone (PCL), Polybutylene succinate (PBS), Polybutylene adipate terephthalate (PBAT), Polyhydroxyalkanoates (PHAs) and Thermoplastic starch (TPS) blends.

The following examples are offered by way of illustration and not by way of limitation.

### EXAMPLE 1

### Strain Construction

The native cutinase-like enzyme (CLE1) gene (Genbank accession number AB671329.1—SEQ ID NO:8), previously known as a lipase from Cryptococcus sp. S-2, was evaluated through an in-silico approach to determine its expression potential in S. cerevisiae. As indicated in Table 1, the native gene has a high GC content (65%), very low codon bias (CBI, 0.07) and codon adaptation indexes (CAI, 0.51) as well as a high number of repeated tandem rare codons (13%). Although these features may be important for proper expression in the native host, these features could have possible negative effects on expression in S. cerevisiae. Transcription can be affected by high GC content; translation efficiency is influenced by codon usage while tandem rare codons may cause ribosomal pausing and bottlenecks during protein secretion. Codon optimisation of the CLE1 gene (SEQ ID NO:1) for expression in S. cerevisiae using the Optimumgene™ algorithm (GeneScript), reduced the GC content (44%), significantly increased the CBI (0.55) and CAI (0.93) and removed tandem rare codons (Table 1).

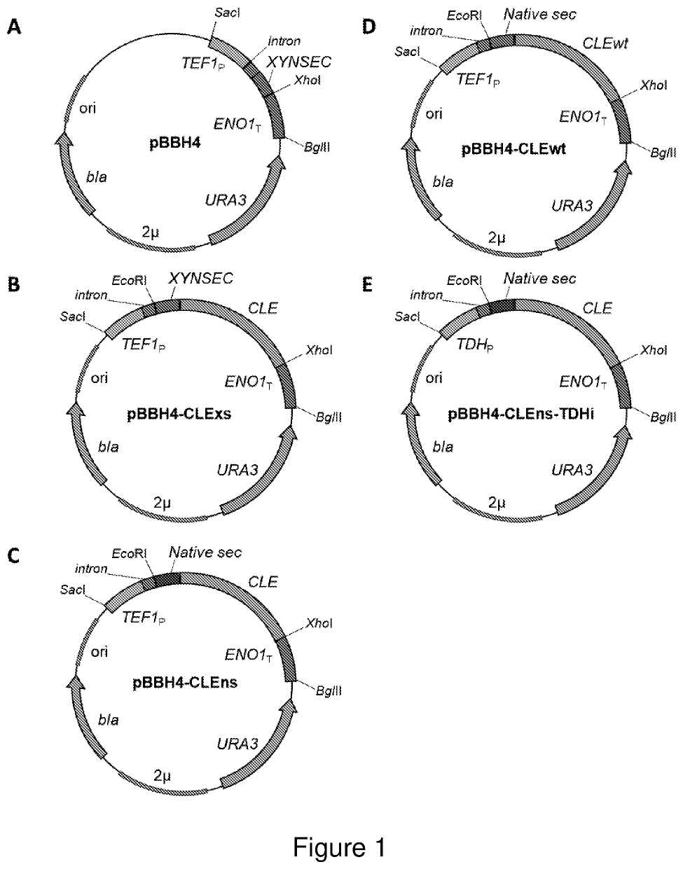

The codon-optimised CLE1 gene, with either its native secretion signal (SEQ ID NO: 3) encoded by a nucleotide sequence of SEQ ID NO:4, or a secretion signal from the Trichoderma reesei xyn2 gene (XYNSEC (SEQ ID NO:5) encoded by a nucleotide sequence of SEQ ID NO:6), was inserted downstream of the TEF1i engineered yeast promoter (SEQ ID NO:7) on the pBBH4 yeast episomal plasmid (FIG. 1A) to deliver the pBBH4-CLExs and pBBH4-CLEns vectors (FIGS. 1B and 1C, Table 2). The engineered TEF1i promoter was constructed and is fully described in Myburgh et al. (FEMS Yeast Res. 2020. 20(6), foaa047), which is incorporated herein by reference in its entirety. This promoter was shown to strongly enhance heterologous expression of an amylase gene in S. cerevisiae. The episomal vectors were transformed into the S. cerevisiae Y294 strain to yield the recombinant Y294[CLExs] and Y294[CLEns] strains.

In a subsequent set of experiments, an additional vector was constructed to compare the expression of the native CLE1 gene with the codon-optimised gene. Another vector was also constructed to compare expression of the codon-optimised CLE1 gene under the control of the engineered TEF1i promoter with the expression under another engineered promoter, TDHi (FIG. 1). This vector was also used in experiments to compare the expression of the native CLE1 gene with the codon-optimised gene.

The native CLE1 gene (SEQ ID NO:8), i.e. not optimised for S. cerevisiae codon usage, with its native secretion signal (SEQ ID NO: 4), was inserted downstream of the engineered TEF1i promoter (SEQ ID NO:7) on the episomal pBBH4 plasmid to yield plasmid pBBH4-CLEwt (FIG. 1D). The codon-optimised CLE1 gene, with its native secretion signal (SEQ ID NO:3) encoded in its entirety by a nucleotide sequence of SEQ ID NO:1 was inserted downstream of the TDHi engineered yeast promoter (SEQ ID NO:9) on the pBBH4 yeast episomal plasmid to deliver the pBBH4-CLEns-TDHi vector (FIG. 1E). The engineered TDHi promoter was constructed and is fully described in Myburgh et al. (FEMS Yeast Res. 2020. 20(6), foaa047), which is incorporated herein by reference in its entirety. As the previous experiments showed that use of the alternative secretion signal XYNSEC (SEQ ID NO:5) did not increase CLE1 protein production, the native secretion signal (SEQ ID NO:3) was used in this subsequent vector. The episomal vectors were transformed into the S. cerevisiae Y294 strain to yield the recombinant Y294[CLEwt] and Y294[CLEns]-TDHi strains (Table 2).

All sequences referred herein to are provided in Table 3.

TABLE 1
In silico evaluation of CLE1 gene expression
potential in S. cerevisiae

| Native Cutinase-like | Codon-optimised Cutinase-like |
|---|---|
| enzyme gene (CLE1) from | enzyme gene (CLE1) from |
| Cryptococcus sp. S-2 | Cryptococcus sp. S-2 |
| GC content - 65% | GC content - 44% |
| Codon bias index (CBI) - 0.07 | Codon bias index (CBI) - 0.55 |
| Codon adaptation | Codon adaptation |
| index (CAI) - 0.51 | index (CAI) - 0.93 |
| Tandem rare codons - 13% | Tandem rare codons - 0% |

TABLE 2
Description of strains and plasmids used herein

| | |
|---|---|
| S. cerevisiae strains | |
| Y294[BBH] | Negative control strain containing empty vector |
| Y294[CLExs] | Y294 strain producing codon-optimised cutinase- |
| | like enzyme from Cryptococcus sp. S-2 with |
| | XYNSEC (SEQ ID NO: 5) secretion signal and |
| | TEF1i promoter (SEQ ID NO: 7) responsible for |
| | expression |
| Y294[CLEns] | Y294 strain producing codon-optimised cutinase- |
| | like enzyme from Cryptococcus sp. S-2 with |
| | native secretion signal (SEQ ID NO: 3) and |
| | TEF1i promoter (SEQ ID NO: 7) responsible |
| | for expression |
| Y294[CLEwt] | Y294 strain producing native cutinase-like |
| | enzyme from Cryptococcus sp. S-2 with native |
| | secretion signal (SEQ ID NO: 3) and TEF1i |
| | promoter (SEQ ID NO: 7) driving expression |
| Y294[CLEns]-TDHi | Y294 strain producing codon optimised cutinase- |
| | like enzyme from Cryptococcus sp. S-2 with |
| | native secretion signal (SEQ ID NO: 3) and |
| | TDHi promoter (SEQ ID NO: 9) driving |
| | expression |
| Commercial enzymes | |
| Proteinase-K (ProK) | Pure lyophilised enzyme from Tritirachium album |
| | Limber (Promega Corporation) used as positive |
| | control |

TABLE 3
Sequences used in the Examples and referred to herein

| Description | Sequence |
|---|---|
| Codon- | ATGTTGGTTTCAGCATTGGCTTTAGCAGTTTTGTCTGCTGC |
| optimised CLE1 | ATCATTAGGTAGAGCTGCACCAACACCAGAATCTGCTGAA |
| gene | GCACATGAATTGGAAGCTAGAGCAACTTCTTCAGCTTGTCC |
| (SEQ ID NO: 1) | ACAATACGTTTTGATTAATACAAGAGGTACTGGTGAACCAC |
| | AAGGTCAATCAGCTGGTTTTAGAACAATGAACTCTCAAATT |
| | ACTGCTGCATTATCAGGTGGTACAATCTATAACACAGTTTA |
| | CACTGCTGATTTCTCTCAAAATTCAGCTGCAGGTACTGCAG |
| | ATATCATCAGAAGAATTAATTCTGGTTTGGCTGCAAACCCA |
| | AACGTTTGTTACATCTTGCAAGGTTACTCACAAGGTGCTGC |
| | AGCTACAGTTGTTGCTTTGCAACAATTAGGTACTTCTGGTG |
| | CAGCTTTTAATGCAGTTAAGGGTGTTTTCTTGATCGGTAAC |
| | CCAGATCATAAGTCTGGTTTGACATGTAACGTTGATTCAAA |
| | TGGTGGTACTACAACTAGAAATGTTAATGGTTTGTCTGTTG |
| | CTTATCAAGGTTCTGTTCCATCAGGTTGGGTTTCAAAAACA |
| | TTAGATGTTTGTGCTTACGGTGACGGTGTTTGTGATACTGC |
| | TCATGGTTTCGGTATTAATGCACAACATTTGTCTTATCCATC |
| | AGATCAAGGTGTTCAAACTATGGGTTACAAGTTCGCTGTTA |
| | ATAAGTTGGGTGGTTCTGCATAA |
| CLE1 | MLVSALALAVLSAASLGRAAPTPESAEAHELEARATSSACPQ |
| (SEQ ID NO: 2) | YVLINTRGTGEPQGQSAGFRTMNSQITAALSGGTIYNTVYTAD |
| | FSQNSAAGTADIIRRINSGLAANPNVCYILQGYSQGAAATVVA |
| | LQQLGTSGAAFNAVKGVFLIGNPDHKSGLTCNVDSNGGTTTR |
| | NVNGLSVAYQGSVPSGWVSKTLDVCAYGDGVCDTAHGFGIN |
| | AQHLSYPSDQGVQTMGYKFAVNKLGGSA |
| Native secretion | MLVSALALAVLSAASLGRA |
| signal of CLE1 | |
| (SEQ ID NO: 3) | |
| Codon- | ATGTTGGTTTCAGCATTGGCTTTAGCAGTTTTGTCTGCTGC |
| optimised native | ATCATTAGGTAGAGCT |
| secretion signal | |
| of CLE1 | |
| (SEQ ID NO: 4) | |
| Secretion signal | MVSFTSLLAGVAAISGVLAAPAAEVEPVAVEKR |
| from | |
| Trichoderma | |
| reesei xyn2 | |
| (SEQ ID NO: 5) | |
| Codon- | ATGGTCTCCTTCACCTCCCTCCTCGCCGGCGTCGCCGCCA |
| optimised | TCTCGGGCGTCTTGGCCGCTCCCGCCGCCGAGGTCGAAC |
| secretion signal | CCGTGGCTGTGGAGAAGCGC |
| from | |
| Trichoderma | |
| reesei xyn2 | |
| (SEQ ID NO: 6) | |
| TEF1i promoter | GCCGTACCACTTCAAAACACCCAAGCACAGCATACTAAATT |
| (SEQ ID NO: 7) | TCCCCTCTTTCTTCCTCTAGGGTGTCGTTAATTACCCGTAC |
| | TAAAGGTTTGGAAAAGAAAAAAGAGACCGCCTCGTTTCTTT |
| | TTCTTCGTCGAAAAAGGCAATAAAAATTTTTATCACGTTTCT |
| | TTTTCTTGAAAATTTTTTTTTTTGATTTTTTTCTCTTTCGATGA |
| | CCTCCCATTGATATTTAAGTTAATAAACGGTCTTCAATTTCT |
| | CAAGTTTCAGTTTCATTTTTCTTGTTCTATTACAACTTTTTTT |
| | ACTTCTTGCTCATTAGAAAGAAAGCATAGCAATCTAATCTAA |
| | GTTTTAATTACAAAGTATGTATCTATAAATTTGAAACCAATAT |
| | TAGGCGGATAAGATGAAATAGTGACTGGCCAATCCAGGAT |
| | TTAATATGTCATAAAAGCCTGTTCTACCTTAATGGGATGAAT |
| | ATCCATGTCTCTGTTATATGCTCCATAGCACAGATCTAGCT |
| | ACCCATGGTACTTTGAGAGAAGGAAACTACGTACTTAGTGG |
| | TGATTTACAGTTAAATTCAATCTACAATTCAAGTCATTAGCA |
| | AATAGTCTTTATCCCAAATTCTACTAGAGTTCGGTTTTTTAC |
| | TAACAAGTATGTTTTTACTTTTTACTTTATCATAGAACATTTA |
| | ATAAATC |
| Native CLE1 | ATGCTCGTCTCCGCTCTCGCTCTCGCGGTGCTGTCCGCTG |
| gene | CTTCTCTCGGCCGAGCCGCACCAACGCCCGAGTCCGCCG |
| (SEQ ID NO: 8) | AGGCGCACGAGCTCGAGGCCCGCGCCACGTCCAGCGCTT |
| | GTCCGCAGTACGTCCTGATCAACACGCGAGGCACGGGCG |
| | AGCCGCAAGGCCAGTCGGCCGGCTTCCGAACGATGAACA |
| | GCCAGATCACCGCCGCGCTGTCGGGTGGCACCATCTACAA |
| | CACTGTCTACACCGCCGATTTCAGCCAGAACAGCGCGGCC |
| | GGCACGGCCGACATCATCCGCCGGATCAACTCGGGTCTC |
| | GCGGCCAACCCGAACGTGTGCTACATCCTCCAAGGGTACA |
| | GCCAGGGCGCGGCTGCTACCGTCGTCGCGCTGCAACAGC |
| | TCGGCACGAGTGGAGCGGCGTTCAACGCCGTCAAGGGTG |
| | TGTTCCTCATTGGCAACCCGGACCACAAGTCGGGCCTGAC |
| | TTGCAACGTCGACTCGAACGGCGGCACTACCACACGCAAT |
| | GTCAACGGCCTGTCGGTCGCGTACCAGGGCTCGGTCCCC |
| | TCAGGATGGGTCAGCAAGACTCTCGATGTCTGCGCTTATG |
| | GCGACGGCGTGTGCGACACCGCGCACGGATTCGGTATCA |
| | ACGCACAGCACCTGTCGTACCCTAGTGACCAAGGCGTCCA |
| | GACCATGGGATACAAGTTTGCCGTCAACAAGCTTGGCGGG |
| | TCGGCCTAA |
| TDHi promoter | AGTTTATCATTATCAATACTGCCATTTCAAAGAATACGTAAA |
| (SEQ ID NO: 9) | TAATTAATAGTAGTGATTTTCCTAACTTTATTTAGTCAAAAAA |
| | TTAGCCTTTTAATTCTGCTGTAACCCGTACATGCCCAAAATA |
| | GGGGGGGGGTTACACAGAATATATAACATCGTAGGTGTCT |
| | GGGTGAACAGTTTATTCCTGGCATCCACTAAATATAATGGA |
| | GCCCGCTTTTTAAGCTGGCATCCAGAAAAAAAAAGAATCCC |
| | AGCACCAAAATATTGTTTTCTTCACCAACCATCAGTTCATAG |
| | GTCCATTCTCTTAGCGCAACTACAGAGAACAGGGGCACAA |
| | ACAGGCAAAAAACGGGCACAACCTCAATGGAGTGATGCAA |
| | CCTGCCTGGAGTAAATGATGACACAAGGCAATTGACCCAC |
| | GCATGTATCTATCTCATTTTCTTACACCTTCTATTACCTTCT |
| | GCTCTCTCTGATTTGGAAAAAGCTGAAAAAAAAGGTTGAAA |
| | CCAGTTCCCTGAAATTATTCCCCTACTTGACTAATAAGTATA |
| | TAAAGACGGTAGGTATTGATTGTAATTCTGTAAATCTATTTC |
| | TTAAACTTCTTAAATTCTACTTTTATAGTTAGTCTTTTTTTTA |
| | GTTTTAAAACACCAAGAACTTAGTTTCGAGTATGTATCTATA |
| | AATTTGAAACCAATATTAGGCGGATAAGATGAAATAGTGAC |
| | TGGCCAATCCAGGATTTAATATGTCATAAAAGCCTGTTCTA |
| | CCTTAATGGGATGAATATCCATGTCTCTGTTATATGCTCCAT |
| | AGCACAGATCTAGCTACCCATGGTACTTTGAGAGAAGGAA |
| | ACTACGTACTTAGTGGTGATTTACAGTTAAATTCAATCTACA |
| | ATTCAAGTCATTAGCAAATAGTCTTTATCCCAAATTCTACTA |
| | GAGTTCGGTTTTTTACTAACAAGTATGTTTTTACTTTTTACTT |
| | TATCATAGAACATTTAATAAATC |

### EXAMPLE 2

### Extracellular CLE1 Production

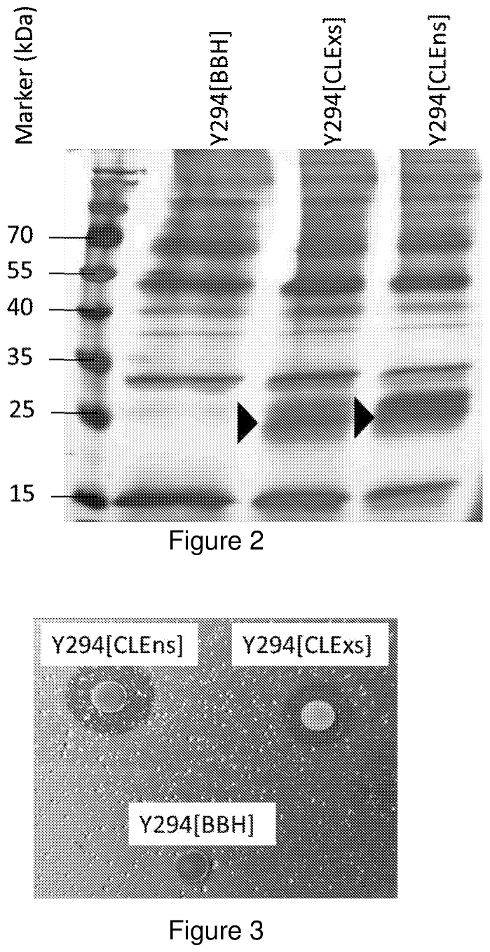

As seen in FIG. 2, SDS-PAGE analysis confirmed the extracellular production of the CLE1 protein by both the Y294[CLExs] and Y294[CLEns] strains. The protein species is easily identified (indicated by black arrow heads), but is larger (25 kDa) than the expected 22 kDa for the CLE1 protein. This is most probably due to glycosylation of the protein species by the yeast cells during the secretion of the enzyme into the extracellular matrix. Indeed, signs of differential glycosylation are evident in samples from both strains, observed as a slight smear or several protein signals for the single CLE1 protein. Glycosylation of enzymes is known to affect their stability, which may be an important factor during treatment of bioplastic materials. Furthermore, it is clear from the intensity of the protein bands that the Y294[CLEns] strain, producing CLE1 with its native secretion signal, delivered more recombinant enzyme in the extracellular fraction than the Y294[CLExs] strain.

FIG. 3 shows hydrolysis halo formation surrounding the Y294[CLEns] and Y294[CLExs] recombinant strains on SC-URA agar plates containing Tributyrin (1% v/v). The Y294[BBH] control strain did not show any halo formation. It is also clear that a larger hydrolysis halo is formed around Y294[CLEns] compared to Y294[CLExs].

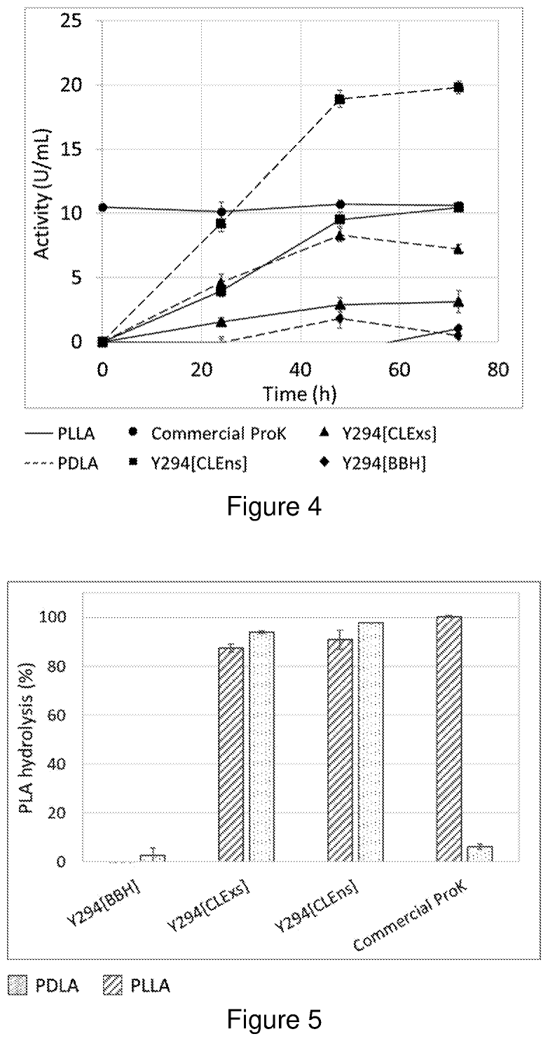

FIG. 4 shows results from turbidity-based enzyme assays, which entails incubating PLLA (represented by solid lines) and PDLA (represented by dashed lines) substrate solutions with supernatant collected from 24, 48 and 72 h yeast cultures.

The ProK positive control showed constant activity over multiple assay rounds on the PLLA substrate. This indicates that the assay is accurate and reproducible across different time points. The Y294[BBH] control showed no significant activity on either PLLA or PDLA substrates, confirming that a decrease in turbidity is due to the effect of the recombinant (or commercial) enzyme and not self-hydrolysis of the PLA polymer. It is evident that the secreted protein species observed during SDS-PAGE analysis (FIG. 2) was active against both the PLLA and PDLA substrates.

The Y294[CLEns] strain delivered substantial activity levels on the PLLA substrate, reaching 10 U/mL after 48 h of cultivation. The Y294[CLExs] strain showed significantly lower activity levels compared to Y294[CLEns], reaching an activity 3.12 U/mL after 72 h cultivation.

Higher activity levels were detected for both recombinant strains on the PDLA substrate compared to PLLA. The Y294[CLEns] and Y294[CLExs] strains reached 20 U/mL and 7.22 U/mL after 72 h, respectively. These results show that active CLE1 enzyme is produced by both recombinant S. cerevisiae strains. However, it is clear that the Y294[CLEns] strain produces higher amounts of extracellular protein that results in better activity levels during the enzyme assays.

### EXAMPLE 3

### Small Scale Cell-Free Hydrolysis on PLA Powder

Small scale cell-free hydrolysis trials were conducted using emulsified PLA substrates with a final concentration of 0.075% w/v incubated with 2.5 mL of cell-free supernatant at 37° C. FIG. 5 depicts the results from the small-scale hydrolysis trials using PLA emulsions. Complete (98%) hydrolysis of emulsified PDLA was achieved after 72 h hydrolysis with supernatant from Y294[CLEns] while near complete (91%) hydrolysis of PLLA was attained by the same strain. The control and commercial ProK did not show any decrease in turbidity of the PDLA substrate, but commercial ProK delivered complete hydrolysis of the PLLA substrate. This indicates a similar time for complete hydrolysis of PLA emulsions compared to a known purified and concentrated CLE1 preparation from the native Cryptococcus sp. S-2 strain. Importantly, a higher concentration of PLA substrate (0.075% w/v) was completely hydrolysed using the present recombinant strains when compared to the known strain (0.04% w/v substrate), which suggests improved hydrolysis when using supernatant as-is (without any purification or concentration) from the recombinant S. cerevisiae strains from this invention. Further strengthening the evidence that the strains of the present invention show improved PLA hydrolysis, is the fact that no surfactants were used during the hydrolysis of emulsified PLA by the strains of the current invention, whereas previous hydrolysis studies included Plysurf A210G in the assays. Surfactants such as Plysurf have been shown to increase PLA hydrolysis by various hydrolases.

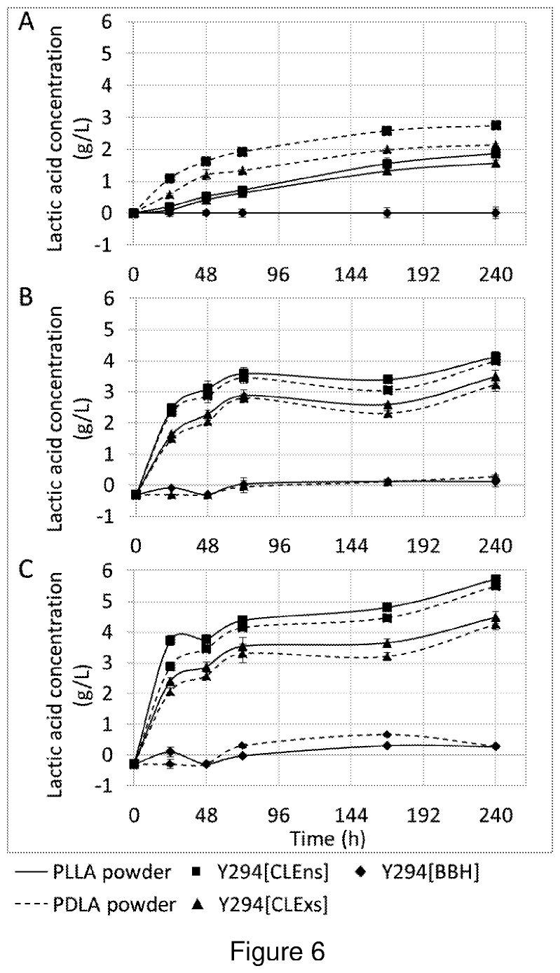

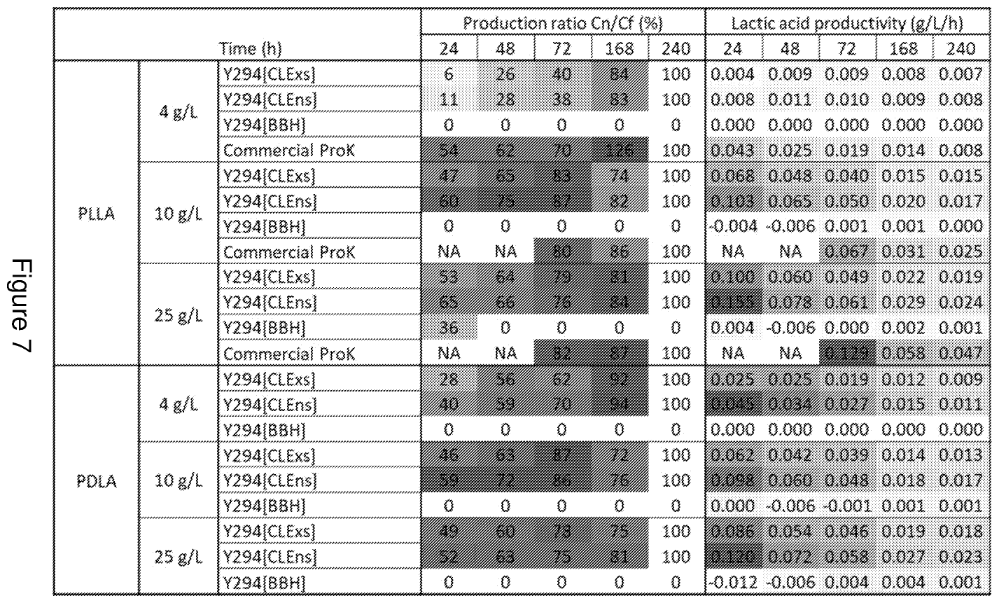

HPLC analysis was conducted on samples collected at several time points during a cell-free hydrolysis trial to illustrate the hydrolysis of PLLA and PDLA powders as reflected in the residual lactic acid concentration. FIG. 6 shows the production of lactic acid during cell-free hydrolysis of PLLA (solid lines) and PDLA (dashed lines) powders using various substrate concentrations (A=4; B=10 and C=25 g/L PLA powder loading). There is substantial production of free lactic acid by samples incubated with the supernatant of the Y294[CLEns] and Y294[CLExs] strains in all experiments conducted. Once again, it is evident that better CLE1 secretion by the Y294[CLEns] strain results in better hydrolysis of the PLA powders compared to the Y294[CLExs] samples. An increase in powder concentration (substrate loading) resulted in increased final concentrations of lactic acid produced from PLLA as well as PDLA polymers. The highest concentrations of lactic acid produced using the supernatant from the Y294[CLEns] strain was 5.73 and 5.50 g/L from 25 g/L PLLA and PDLA powder, respectively (FIG. 6C).

FIG. 7 shows the ratio of lactic acid produced at each time point relative to the final concentration (%) as well as lactic acid productivity (g/L/h). Shading indicates performance of the strains at each time point (darker shading=better performance). Considering the concentration of lactic acid produced at each time point (Cn) relative to the final concentration of lactic acid produced (Cf) (represented as a percentage in FIG. 7), significantly higher fractions of the final lactic acid concentration were produced from 10 g/L PLA powder compared to 4 and 25 g/L. From 10 g/L PLLA powder, 87% of the final lactic acid concentration was produced within the first 72 h of hydrolysis. Similarly, 86% of the final lactic acid concentration was produced in the same time using 10 g/L PDLA powder. This indicates that most of the lactic acid is produced within 72 h. Lactic acid productivities for hydrolysis trials using Y294[CLEns] supernatant on PLLA and PDLA powders showed maximum rates of 0.155 and 0.120 g/L/h, respectively. This data shows that free lactic acid is produced using the crude enzyme extract from the recombinant strains and that substrate loading influences the efficiency of PLA hydrolysis.

### EXAMPLE 4

### Small Scale Cell-Free Hydrolysis on PLA Film

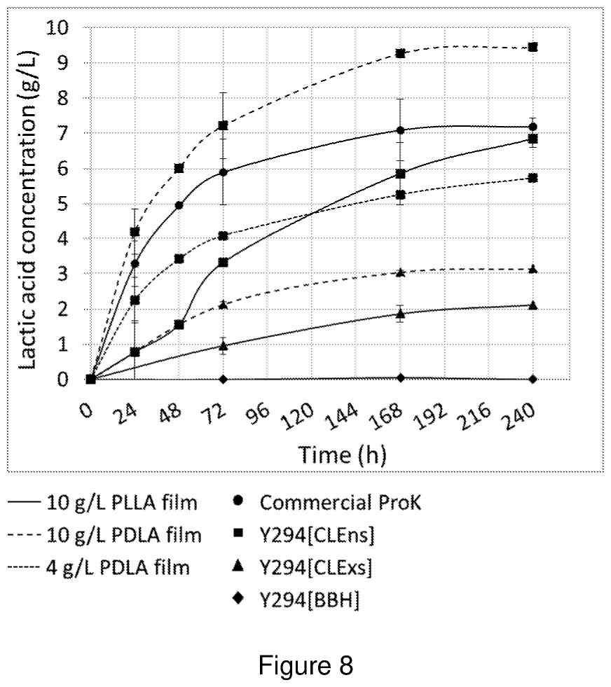

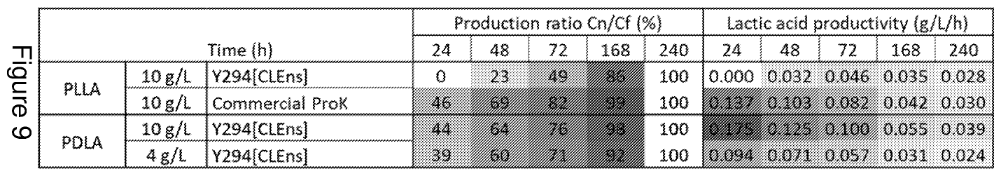

PLA thin films provide a better representation of real-world PLA substrates. Therefore, small scale hydrolysis trials using 10 mL cell-free supernatant (crude enzyme) incubated with PLA films was carried out. FIG. 8 shows lactic acid release from PLLA (solid lines) and PDLA (dashed and dotted lines) films over a 10-day hydrolysis period at 37° C.

Substantial amounts of free lactic acid were produced from 10 g/L PLLA and PDLA films. Surprisingly, the hydrolysis of PLA films resulted in increased levels of lactic acid release compared to powdered PLA (FIG. 8). Powdered substrates have a larger surface area and better enzyme hydrolysis is expected than from film. At the end of the hydrolysis period (240 h), samples treated with supernatant from the Y294[CLEns] strain yielded 6.84 and 9.44 g/L lactic acid from 10 g/L PLLA and PDLA films, respectively. This counter-intuitive result could be due to more hydrophobic surfaces on powder particles being created through processing of the PLA material. The enhanced effect observed throughout previous experiments for the Y294[CLEns] strain than with Y294[CLExs] was even more pronounced in experiments using PLA films, probably due to better secretion and thus higher enzyme concentrations in the extracellular fraction. Although higher final lactic acid concentrations were achieved from the hydrolysis of PLA films, lower lactic acid production ratios (Cn/Cf) (represented as a percentage in FIG. 9) were observed within the first 72 h of hydrolysis compared to using powders. Nonetheless, 49% and 76% of the final lactic acid concentrations were produced in the first 72 h of PLLA and PDLA film hydrolysis, respectively. Furthermore, higher lactic acid productivities were reported from PDLA films with 0.175 g/L/h lactic acid produced from 10 g/L PDLA films in the first 24 h of hydrolysis.

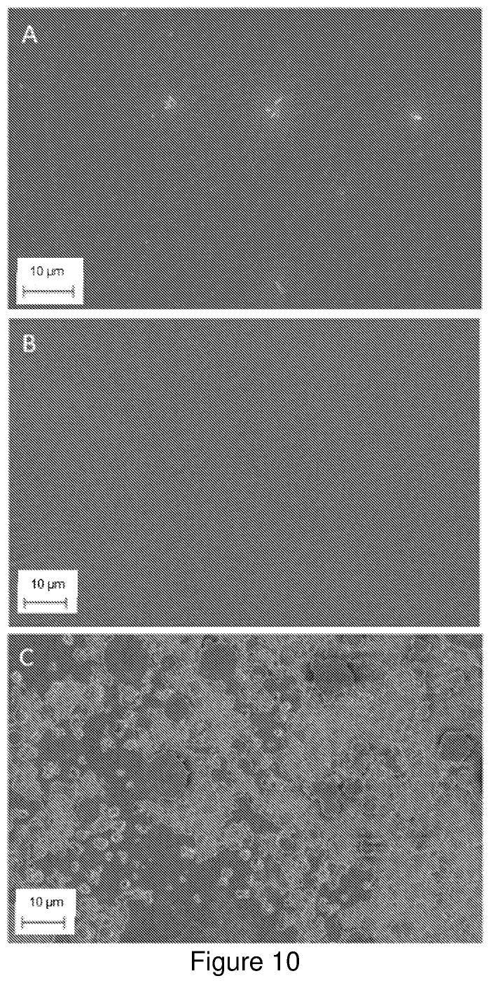

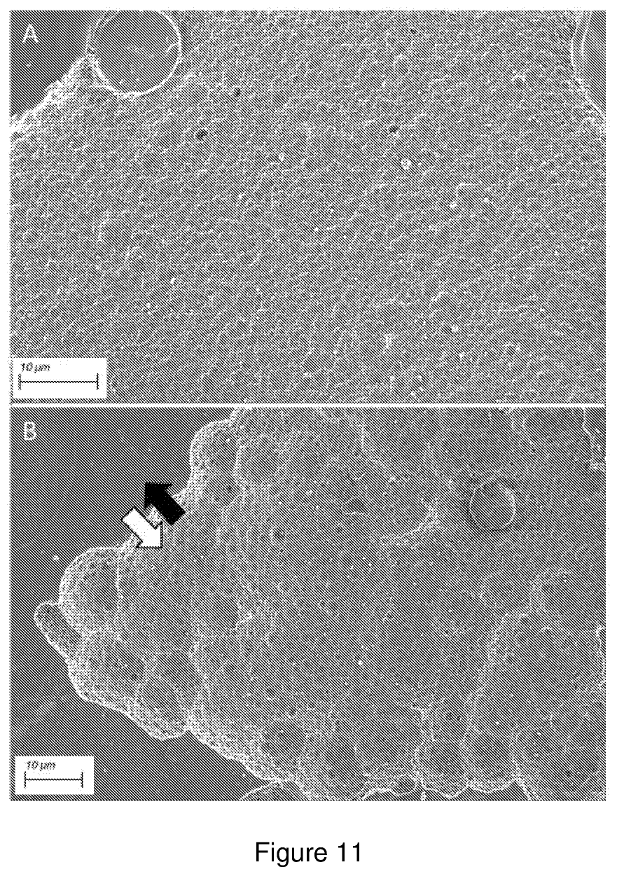

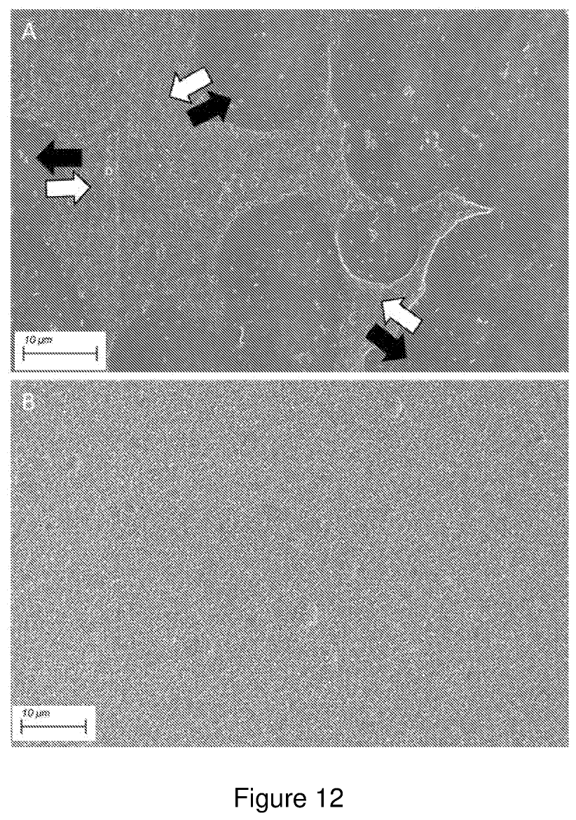

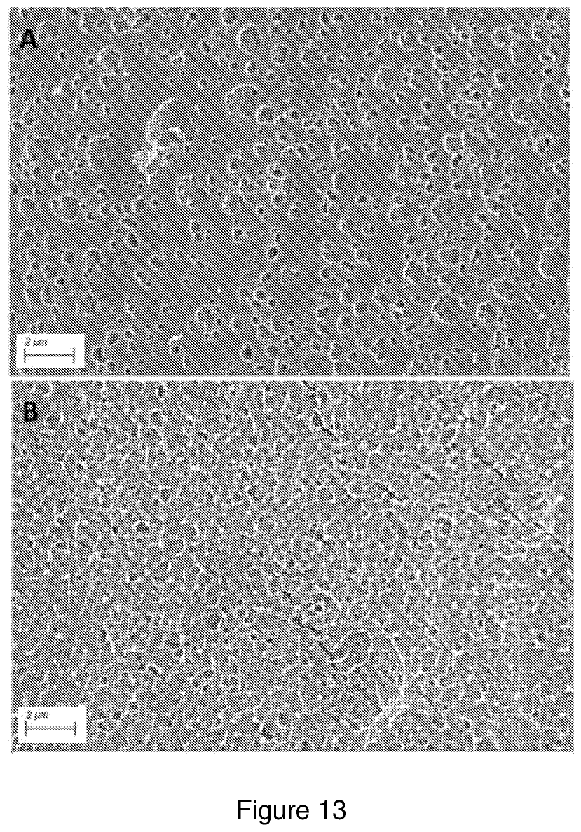

Scanning electron microscopy (SEM) was carried out on PLA films incubated with cell-free supernatant from Y294[CLEns] strain in order to discern specific hydrolysis patterns. SEM analysis of PLA films treated with supernatant from Y294[CLEns] showed clear signs of degradation (FIG. 10C), but no degradation pattern was evident in films incubated with supernatant from the Y294[BBH] control strain (FIG. 10B). Degradation patterns were evident on PDLA films after 24 h hydrolysis and more pronounced after 48 h (FIGS. 11A and 11B, respectively). Hydrolysis started in certain regions of the films and steadily extended outwards to include larger areas as well as inwards into the matrix of the film (white arrow in FIG. 11B). Although PLLA film degradation in general was lower than for PDLA films, specific pit formation was more evident when PLLA films were treated with supernatant from the Y294[CLEns] strain (FIGS. 12A and 13A).

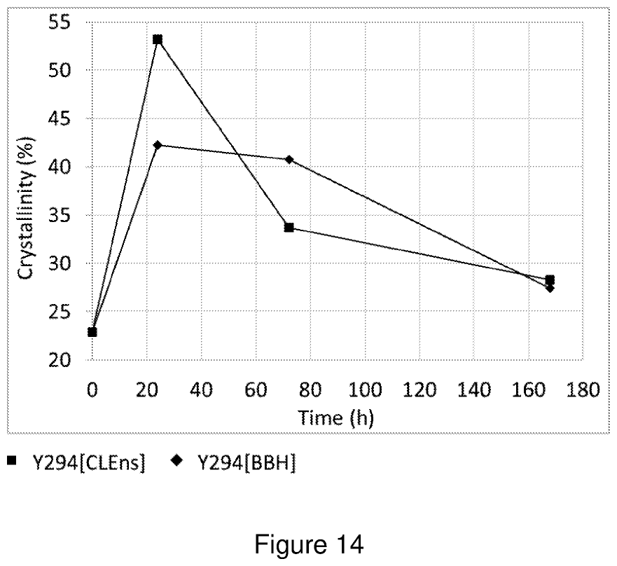

It is also clear that hydrolysis started in amorphous regions (white arrows in FIGS. 11A and 12A) before moving to more crystalline areas (black arrows in FIGS. 11A and 12A), which show no degradation after 24 h (FIG. 12). This is in contrast to a more general hydrolysis pattern observed for PLLA films incubated with commercial ProK (FIGS. 12B and 13B). It appears that the commercial enzyme hydrolyses the film surface at the same rate rather than preferentially hydrolysing amorphous regions (FIGS. 12B and 13B). Hydrolysis of amorphous regions prior to crystalline regions results in an initial increase in crystallinity of the film (i.e. film becomes more brittle) and therefore fragmentation of the film could be more pronounced during this initial hydrolysis. This observation was confirmed through DSC analysis of PLLA film collected at 24 h intervals throughout hydrolysis with supernatant from Y294[CLEns] (FIG. 14). An initial increase in crystallinity in the first 24 h indicates amorphous regions being hydrolysed first, followed by a major decrease in crystallinity at 72 h due to the hydrolysis of crystalline areas. This confirms the mechanism of PLA film hydrolysis by CLE1 observed during SEM.

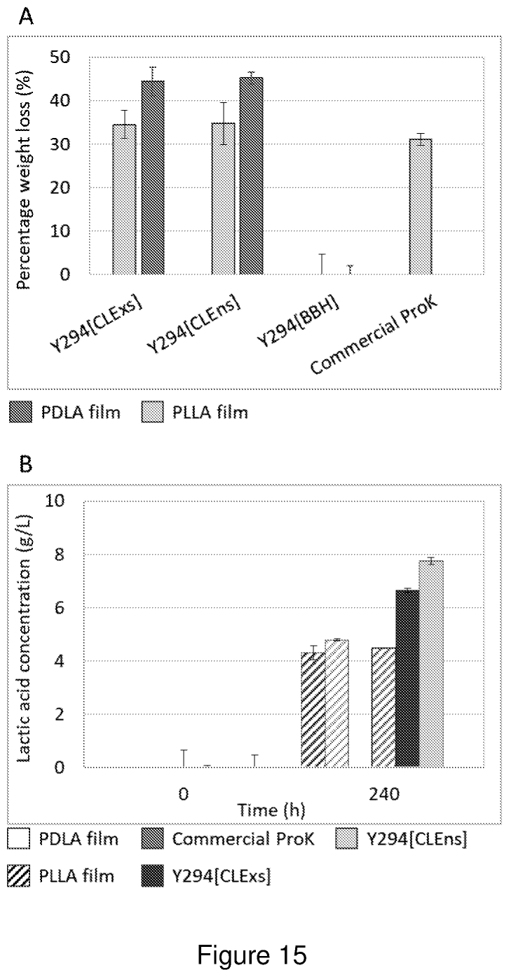

Small-scale film hydrolysis was conducted to determine weight loss of the films after 240 h hydrolysis using 10 mL reaction volumes (10 g/L thin films). Briefly, the PLA film was weighed prior to adding enzyme, filtered through Whatmann filter paper after hydrolysis, and then the total sample was weighed again after drying. HPLC analysis was conducted to determine the final lactic acid concentration produced from hydrolysis. Films treated with each of the recombinant strains' supernatant showed significant weight loss at the end of the hydrolysis period (FIG. 15A). Final weight loss of roughly 35 and 45% were reached for PLLA and PDLA film samples, respectively, relative to the BBH control. This is higher than the 31% weight loss reported for the commercial ProK enzyme.

Once again, substantial lactic acid production was observed from 10 g/L film hydrolysis for both recombinant strains and both PLLA and PDLA films. Final lactic acid concentrations of 4.30 and 4.79 g/L were produced following incubation of PLLA with Y294[CLExs] and Y294[CLEns] supernatant samples respectively (FIG. 15B). While final lactic acid concentrations of 6.64 and 7.75 g/L were produced following incubation of PDLA with Y294[CLExs] and Y294[CLEns] supernatant samples, respectively (FIG. 15B). To the inventors' knowledge, this is the first report showing such extensive film hydrolysis using recombinant S. cerevisiae strains expressing the CLE1 enzyme.

### EXAMPLE 5

### Scaled Up Cell-Free Hydrolysis on PLA Film

The hydrolysis reactions were scaled up to 50 mL working volumes. Substrate loading of 10 g/L was maintained for these experiments. In addition to the lower molecular weight PDLA and PLLA films, two higher molecular weight PLA polymers (PDLAHMW and PLLAHMW) were included in these experiments.

The scale-up of the hydrolysis reactions resulted in similar trends to those observed with the small-scale experiments. Films incubated with supernatant from the Y294[BBH] control strain showed little to no signs of degradation, with the higher molecular weight PLLAHMW and PDLAHMW polymers showing less signs of degradation than the lower molecular weight PLLA and PDLA samples (FIG. 16). The lower molecular weight PLLA and PDLA films treated with supernatant from the Y294[CLEns] strain showed extreme fragmentation after 240 h hydrolysis, with less fragmentation observed for the higher molecular weight PLLAHMW and PDLAHMW polymers, but clearly more than for the Y294[BBH] control.

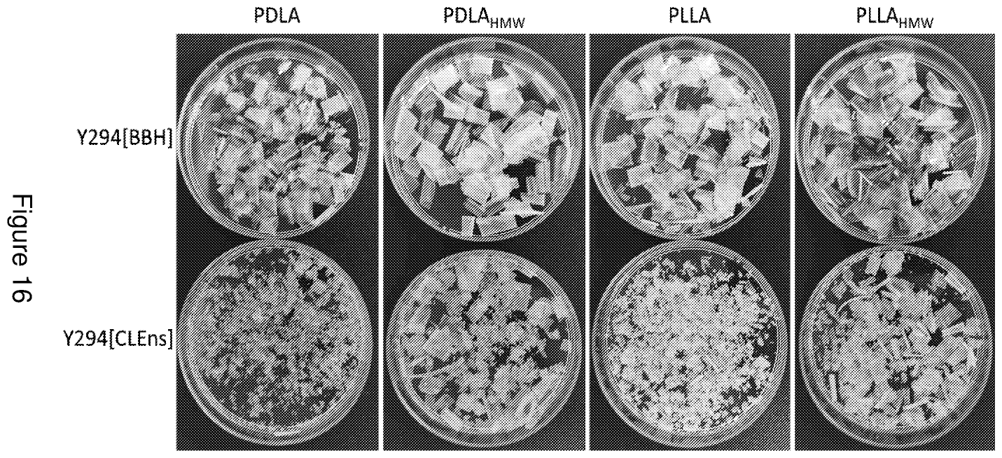

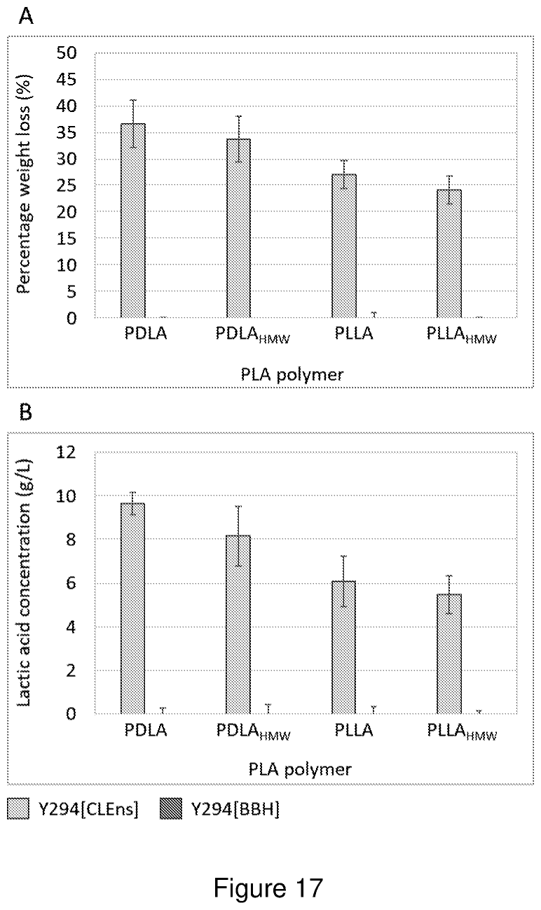

Weight loss data of the two PDLA polymers showed surprisingly similar weight loss percentages for the PDLA and PDLAHMW samples (37 and 34%, respectively) and also for the PLLA and PLLAHMW polymers (27 and 24%, respectively) (FIG. 17A) even though there is increased fragmentation for the lower molecular weight polymer as seen in FIG. 16. Lactic acid concentrations were similar to that observed during small-scale hydrolysis of films (FIG. 17B). As expected, the highest concentration of lactic acid was produced from the PDLA films (9.66 g/L), followed by PDLAHMW (8.16 g/L), PLLA (6.09 g/L) and PLLAHMW (5.48 g/L). It is interesting to note that higher concentrations of lactic acid were produced from the higher molecular weight PDLAHMW polymer than the lower molecular weight PLLA material.

Work reported herein provides comprehensive data on the construction of a recombinant S. cerevisiae strain that produces extracellular CLE1 at high levels allowing for the enhanced degradation of PLA by implementing the extracellular fraction without any need for purification, concentration, or surfactant supplementation. This is the first report showing such positive results for the hydrolysis of PLA materials using a recombinant S. cerevisiae strain to produce an active agent used during PLA degradation. Furthermore, it also provides the most thorough evaluation of using recombinantly produced CLE1 in hydrolysis trials in which decreases in turbidity, lactic acid production and film weight loss all suggest an improved system for PLA degradation. Insight into the mechanism of PLA film hydrolysis is also provided.

### EXAMPLE 6

### Extracellular CLE1 Production

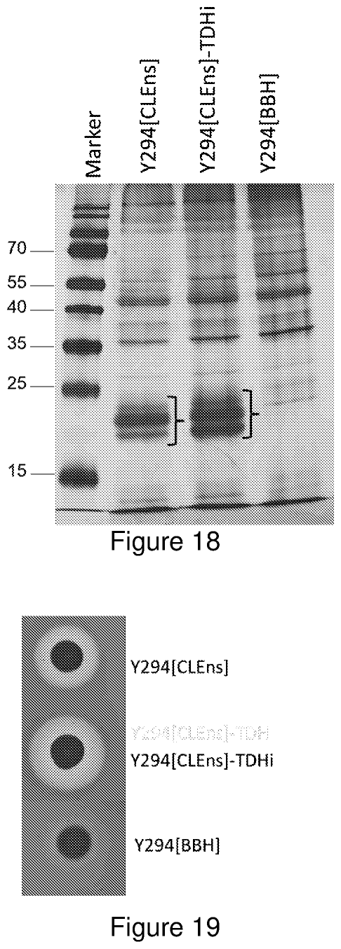

As seen in FIG. 18, SDS-PAGE analysis confirmed the extracellular production of the CLE1 protein by both the Y294[CLEns] and Y294[CLEns]-TDHi strains. The protein species is easily identified (indicated by black arrow heads) and it is clear from the intensity of the protein bands that the Y294[CLEns]-TDHi strain, with expression driven by the engineered TDHi promoter, delivered more recombinant enzyme in the extracellular fraction than the Y294[CLEns] strain.

FIG. 19 shows hydrolysis halo formation surrounding the Y294[CLEns] and Y294[CLEns]-TDHi recombinant strains on SC-URA agar plates containing 0.035 g/L Polycaprolactone (Mw 80 000). The Y294[BBH] control strain did not show any halo formation, while both Y294[CLEns] and Y294[CLEns]-TDHi produced hydrolysis halos. It is also clear that a larger hydrolysis halo is formed around Y294[CLEns]-TDHi compared to Y294[CLEns].

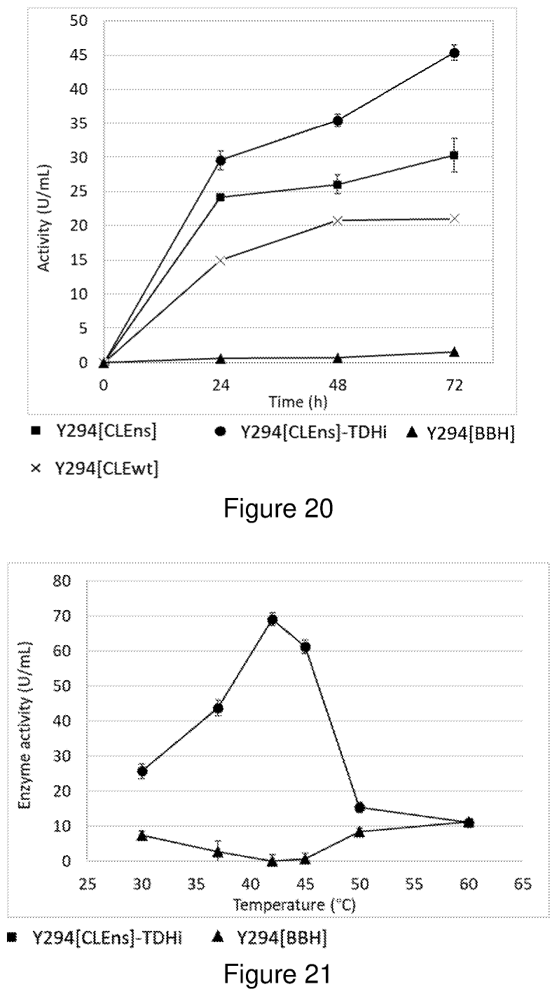

FIG. 20 shows results from turbidity-based enzyme assays. The assay entails incubating emulsified PDLA substrate solutions with supernatant collected from 24, 48 and 72 h yeast cultures. The Y294[BBH] control showed no significant activity, confirming that any decrease in turbidity under the assay conditions is due to the effect of the recombinant enzyme and not self-hydrolysis of the PLA polymer. All three strains expressing CLE1 under the control of engineered promoters showed activity, with both strains including the codon-optimised sequence showing increased activity at all time points. The Y294[CLEwt] strain reached a maximum activity of 21 U/mL which was 10 U/mL less than that detected for the Y294[CLEns] strain. Y294[CLEns]-TDHi was the top-performing strain reaching 45 U/mL after 72 h, a 1.5-fold increase compared to Y294[CLEns], showing the benefit of using the TDHi engineered promoter for the expression of the codon-optimised CLE1 gene.

The optimal temperature for PLA hydrolysis was investigated by conducting turbidity-based assays at various incubation temperatures using supernatant from the Y294[CLEns]-TDHi strain (FIG. 21), which appears to be the best performing strain from the previous results. It is clear that the recombinant CLE1 produced by the Y294[CLEns]-TDHi strain has better PLA hydrolytic activity (1.6-fold increase) at 42° C. than the previously reported optimum of 37° C. Conducting the enzymatic hydrolysis at temperatures approaching the glass transition (Tg) temperature of PLA can improve enzymatic recycling of solid PLA films significantly.

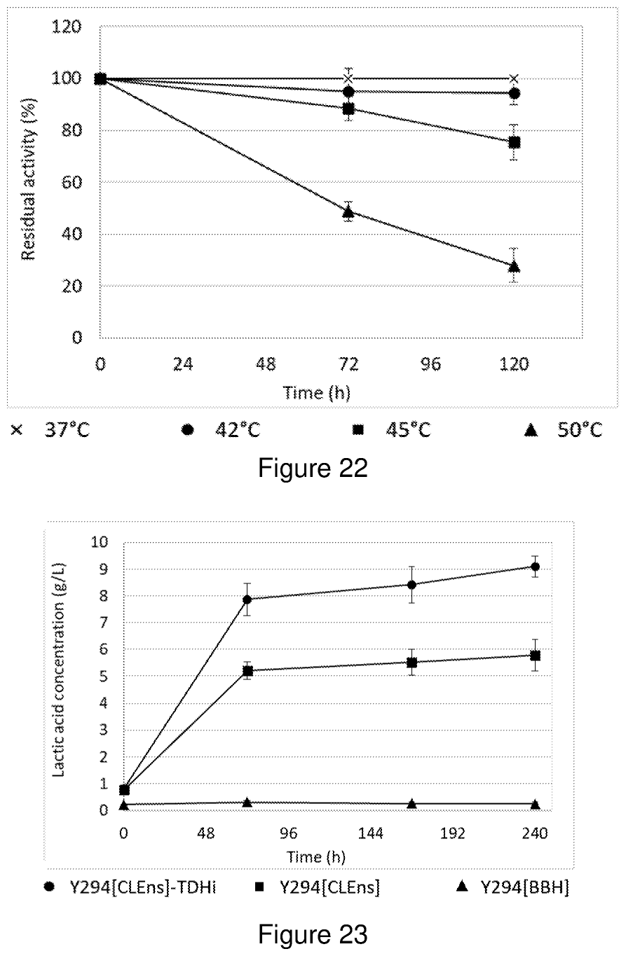

The stability of the recombinant CLE1 species in the supernatant of the Y294[CLEns]-TDHi strain was investigated by incubating supernatant at 37, 42, 45 and 50° C. for 120 h. The enzyme activity remained very stable at 37° C. (100% residual activity) and 42° C. (94% residual activity) while a decrease in residual activity was detected after 120 h incubation at 45 and 50° C., decreasing to 75 and 28% respectively (FIG. 22). Thus, with the recombinant enzyme being highly stable at 42° C., it is a feasible option to conduct PLA film hydrolysis at this temperature.

### EXAMPLE 7

### Small Scale Cell-Free Hydrolysis on PLA Film

PLA thin films provide a better representation of real-world PLA substrates. Therefore, small scale hydrolysis trials using 10 mL supernatant and 10 g/L PLA films was carried out at 37° C. FIG. 23 shows lactic acid released from PLLA films over a 10-day hydrolysis period.

Substantial amounts of free lactic acid were released from 10 g/L PLLA films. After 72 h hydrolysis, samples treated with supernatant from the Y294[CLEns] and Y294[CLEns]-TDHi strains yielded 5.22 and 7.87 g/L lactic acid, respectively. At the end of hydrolysis (240 h), treatment with supernatant from the Y294[CLEns]-TDHi strain delivered 9.10 g/L lactic acid which was 3.31 g/L more than that obtained using supernatant from the Y294[CLEns] strain.

### EXAMPLE 8

### Scaled Up Cell-Free Hydrolysis on PLA Film and Process Improvement

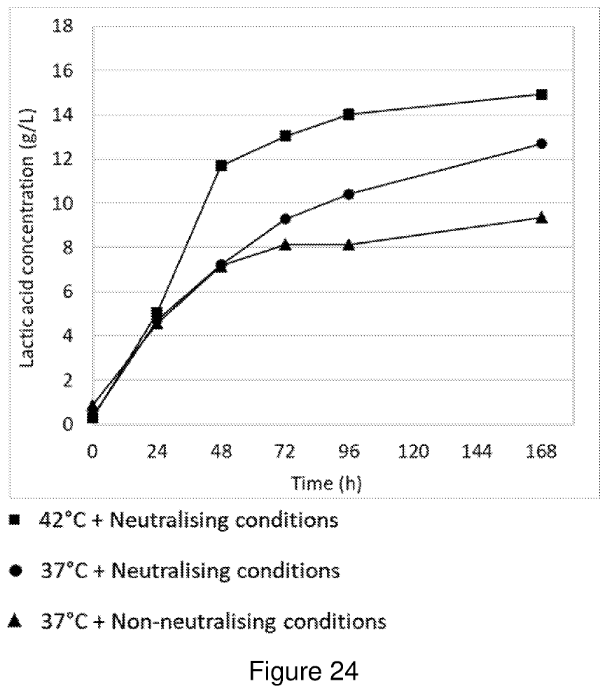

The PLA hydrolysis reactions were scaled up to 700 mL working volumes in controlled bioreactor set-ups using supernatant from the best performing Y294[CLEns]-TDHi strain. A PLA film loading of 10 g/L was used for these experiments while different temperatures (37° C. and 42° C.) and the effect of pH control was investigated. Agitation of the hydrolysis mixture was maintained at 200 rpm with a central impeller and the addition of 3M KOH was used in set-ups requiring pH control. The pH was set to 6.8 and maintained between 6.8 and 7 through a hysteresis setting of 0.2. Lactic acid release was monitored using HPLC analysis at 24 h intervals for a total of 168 h (FIG. 24).

Similar lactic acid release was observed for all three set-ups in the first 24 h of hydrolysis. The advantage of performing the hydrolysis at higher temperature is evident in the release of 4.5 g/L more lactic acid after 48 h at 42° C. compared to 37° C. From 72 h onwards, the benefit of pH control is also evident, as lactic acid release increased more in set-ups with pH control than set-ups without the addition of base. Ultimately, the best conditions for PLA film hydrolysis using the recombinant CLE1 enzyme was found to be at 42° C. and with pH control at 6.8-7. Under these conditions 14.9 g/L lactic acid was released after 168 h hydrolysis which resulted in a 78% weight loss in PLA film.

1. A method for producing a cutinase-like enzyme (CLE1) in a S. cerevisiae cell, the method comprising:
heterologously expressing a nucleic acid encoding the CLE1 in the cell, wherein the nucleic acid encoding the CLE1 is codon-optimised for expression in S. cerevisiae, further wherein the nucleic acid encoding the CLE1 is operably linked to an engineered promoter.

2. The method of claim 1, wherein the cutinase-like enzyme has the amino acid sequence of SEQ ID NO:2.

3. The method of claim 1, wherein the nucleic acid encoding the CLE1 has a nucleotide sequence substantially identical to SEQ ID NO:1.

4. The method of claim 1, wherein the engineered promoter is a TDHi engineered promoter having a nucleotide sequence substantially identical to SEQ ID NO:9 or a TEF1i engineered promoter having a nucleotide sequence substantially identical to SEQ ID NO:7.

5. The method of claim 1, wherein the CLE1 comprises a secretion signal.

6. The method of claim 1, wherein the S. cerevisiae cell is of the strain S. cerevisiae Y294, S. cerevisiae Ethanol Red V1, S. cerevisiae M2n or S. cerevisiae Y130.

7. The method of claim 1, further comprising culturing the S. cerevisiae cell to obtain a population of S. cerevisiae cells.

8. The method of claim 7, further comprising preparing a cell-free supernatant from the population of S. cerevisiae cells, wherein the cell-free supernatant comprises the CLE1 enzyme.

9. A recombinant S. cerevisiae cell comprising:
a nucleic acid encoding a cutinase-like enzyme (CLE1), wherein the nucleic acid encoding the CLE1 is codon-optimised for expression in S. cerevisiae, further wherein the nucleic acid encoding the CLE1 is operably linked to an engineered promoter,
wherein the recombinant S. cerevisiae cell is capable of heterologously expressing the CLE1.

10. The recombinant S. cerevisiae cell of claim 9, wherein the cutinase-like enzyme has the amino acid sequence of SEQ ID NO:2.

11. The recombinant S. cerevisiae cell of claim 9, wherein the nucleic acid encoding the CLE1 has a nucleotide sequence substantially identical to SEQ ID NO:1.

12. The recombinant S. cerevisiae cell of claim 9, wherein the engineered promoter is a TDHi engineered promoter having a nucleotide sequence substantially identical to SEQ ID NO:9 or a TEF1i engineered promoter having a nucleotide sequence substantially identical to SEQ ID NO:7.

13. The recombinant S. cerevisiae cell of claim 9, wherein the CLE1 comprises a secretion signal.

14. The recombinant S. cerevisiae cell of claim 9, wherein the recombinant S. cerevisiae cell is of the strain S. cerevisiae Y294, S. cerevisiae Ethanol Red V1, S. cerevisiae M2n or S. cerevisiae Y130.

15. A cutinase-like enzyme produced by the method of claim 1.

16. A method of preparing a cell-free supernatant comprising a cutinase-like enzyme, the method comprising:
culturing the recombinant S. cerevisiae cell of claim 9 to obtain a population of S. cerevisiae cells; and
preparing a cell-free supernatant from the population of S. cerevisiae cells.

17. The method of claim 16, further comprising concentrating the cell-free supernatant.

18. The method of claim 17, wherein the concentrating is by lyophilisation, filtration, precipitation and/or chromatography.

19. A cell-free supernatant prepared by the method of claim 16.

20. Use of a cutinase-like enzyme of claim 15 for hydrolysing a bioplastic polymer.

21. A method of hydrolysing a bioplastic polymer, comprising incubating the polymer with a cutinase-like enzyme of claim 15.

22. The method of claim 21, wherein the incubating the polymer with the cutinase-like enzyme or the cell-free supernatant is between about 42° C. and about 45° C. and at a pH of 6.8-7.

23. A method of hydrolysing a bioplastic polymer, comprising incubating the polymer with a recombinant S. cerevisiae cell of claim 9.
